(19) United States
(12) Patent Application Publication
(10) Pub. No.: US 2023/0034007 A1
(43) Pub. Date: Feb. 2, 2023

(54) METHODS OF PRESERVING TISSUES FOR TRANSPLANTATION

(71) Applicant: The Regents of the University of California, Oakland, CA (US)

(72) Inventor: Koichi Masuda, La Jolla, CA (US)

(21) Appl. No.: 17/791,187

(22) PCT Filed: Jan. 13, 2021
(86) PCT No.: PCT/US21/13195

§ 371 Date: Jul. 6, 2022

Related U.S. Application Data
(60) Provisional application No. 62/960,298, filed on Jan. 13, 2020.

(51) Int. Cl. A01N 1/02; C12M 3/00; C12M 1/00; C12M 1/12; C12N 5/071; C12N 5/077

(52) U.S. Cl. CPC A01N 1/0231; A01N 1/0226; A01N 1/0252; C12M 21/08; C12M 41/14; C12M 25/02; C12N 5/0697; C12N 5/0654; C12N 5/0655

(57) ABSTRACT

Provided herein are systems and methods for preserving and transporting tissues for transplantation. In particular, tissue to be implanted is encapsulated in an alginate gel, which is exposed to culture medium to improve cell viability and suppress inflammatory factors during long-term storage and transport.

### CROSS-REFERENCE TO RELATED APPLICATION

This application claims priority under 35 U.S.C. § 119(e) to U.S. Provisional Application No. 62/960,298, filed Jan. 13, 2020, the content of which is hereby incorporated by reference in its entirety.

### TECHNICAL FIELD

The disclosure relates generally to the field of tissue, such as an allograft, and more specifically to long-term tissue storage and preservation for transplantation.

### BACKGROUND

Osteochondral allografts (OCA) allow transplantation of viable, functional tissue for treatment of cartilage defects without the need for immunosuppression. Thus, OCA is one of the most widely used methods applied for the repair of middle to large articular cartilage defects (Mandelbaum, et al., Am J Sports Med. 1998; 853). Recent clinical results showed that OCAs were successful in 60% to 80% of cases at 10 years postoperatively (Cavendish, et al., JBJS Rev. 2019; e7). These grafts are procured from organ donors and must be stored to allow for viral and bacterial testing for safety prior to shipping to surgical centers for implantation into patients. Based on studies looking at viability of the cells in the grafts, recommendations have been given for implanting tissues as soon after harvest as possible in order to maximize success.

One of the key factors for obtaining satisfactory clinical results is to improve the quality of stored OCAs, including maximizing chondrocyte viability and cartilage matrix content. Although various studies have previously been performed to improve storage conditions, the quality of grafts stored for long periods of time remains unsatisfactory (Ball, et al., Clin Orthop Relat Res. 2004:246; Williams, et al., J Bone Joint Surg. Am. 2003:2111; and Pallante, et al., Am J Sports Med. 2009:24S). The improvement of storage conditions will increase the shelf life of OCAs and the availability of their clinical usage with an improved clinical outcome.

### SUMMARY OF THE DISCLOSURE

The present disclosure is based on the discovery that encapsulation of tissue to be implanted in alginate gel improves the quality and viability of the tissue after long-term term storage, or transportation, both storage and transportation.

Accordingly, the disclosure provides a method for osteochondral tissue preservation or preservation of other tissue(s) or organ(s), such as an articular cartilage free of any subchondral bone, a fibrocartilage tissue (such as a meniscus, e.g., a medial meniscus or a lateral meniscus or both), a tendon (such as a patellar tendon or an Achilles tendon), a cornea, or a cornea with a vitreous body. In various embodiments, the osteochondral tissue comprises, consists essentially of, or consists of an osteochondral core trimmed to have a bone-to-cartilage-thickness ratio of from about 20:1 to about 1:10 and ranges or ratios in between, for example, about 4:1. The method includes (for example, comprises, or consists essentially of, or further consists of) encapsulating the osteochondral tissue or other suitable tissue(s) or organ(s) in an alginate gel and exposing the gel-encapsulated osteochondral tissue or other suitable tissue(s) or organ(s) to a culture medium. In some embodiments, the gel-encapsulated tissue is in the culture medium for about 24 hours to 8 weeks, or for at least about 2 weeks, such as at least about 4 weeks, at least about 6 weeks, or longer and ranges in between. In various embodiments, the gel-encapsulated tissue in the culture medium is then used for implantation. In various embodiments, the method further comprises encapsulating the tissue(s) or organ(s) in a semipermeable membrane. In various embodiments, the semipermeable membrane has a feature of one or more of: a pore size of at least about 250 pm, a pore size of about 0.22 μm to about 1000 μm and ranges in between, such as about 0.22 μm to about 100 μm or about 0.22 μm to about 100 Additionally or alternatively, the semipermeable membrane has a feature of a molecular weight cut-off (MWCO) at about 50 kDa or at about 200,000 kDa. Additionally or alternatively, the semipermeable membrane has a mesh opening rate of about 1% to about 50% and ranges or percentages in between, such as 2%. In various embodiments, the tissue is encapsulated in the gel prior to, during, or after being encapsulated in the semipermeable membrane. In various embodiments, the alginate gel is ultra-purified alginate (UPAL) gel. In various embodiments, the alginate, such as UPAL, is present in the gel at a concentration of about 0.5% to about 2.0% and ranges or percentages in between, for example, about 1.2%. In various embodiments, the culture medium is complete medium. In various embodiments, the culture medium is not replenished after exposing the gel-encapsulated osteochondral tissue or other gel-encapsulated tissue(s) or organ(s) to the culture medium. In various embodiments, the method further comprises exposing the tissue(s) or organ(s) to a temperature below about 10° C., for example, from about 0° C. to about 4° C. ranges in between, or at about 4° C. In various embodiments, the encapsulated tissue(s) or organ(s) is or are not stored with one or more of: an artificially controlled CO₂ level, an artificially controlled 02 level, or an artificially controlled humidity. In various embodiments, the method further comprises dissolving the alginate gel after the exposure with the culture medium. In further embodiments, the alginate gel is dissolved by a method comprising, or consisting essentially or, or consisting of, exposing the encapsulated tissue(s) or organ(s) to a chelating solution. In yet further embodiments, the chelating solution comprises, consists essentially of, or further consists of a sodium citrate solution or an Ethylenediaminetetraacetic acid (EDTA) solution or a solution comprising or consisting essentially of a sodium citrate and an EDTA. In various embodiments, the method further comprises disassembling the semipermeable membrane after the exposure with the culture medium, for example, mechanically or by a chelating solution. In various embodiments, the encapsulated tissue(s) or organ(s) reduces or reduce release of inflammatory factor(s), for example, compared to a tissue or organ stored without the encapsulation. In further embodiments, the inflammatory factor(s) comprises, consists essentially of, or further consists of nitric oxide, one or more of cytokines, or both. In various embodiments, the encapsulated tissue(s) or organ(s) prior to the implantation maintains or maintain a cell viability of at least about 60%, or at least about 65%, or at least about 70%, or at least about 75%, or at least about 80%, or at least about 85% or higher of that prior to the encapsulating step. In various embodiments, prior to exposing to the culture medium, the encapsulated tissue(s) or organ(s) is or are washed with a buffer, such as a saline. In various embodiments, the encapsulated tissue(s) or organ(s), (e.g., cornea) is in the culture medium for at least about 2 weeks.

In another aspect, the disclosure provides a method for storing osteochondral tissue or other tissue(s) or organ(s), such as an articular cartilage free of any subchondral bone, a fibrocartilage tissue (such as a meniscus, e.g., a medial meniscus or a lateral meniscus or both), a tendon (such as a patellar tendon or an Achilles tendon), a cornea, or a cornea with a vitreous body for example, prior to implantation. In various embodiments, the osteochondral tissue comprises, consists essentially of, or consists of an osteochondral core trimmed to have a bone-to-cartilage-thickness ratio of from about 20:1 to about 1:10, for example, about 4:1. The method includes (for example, comprises, or consists essentially of, or further consists of) optionally obtaining osteochondral tissue or other suitable tissue(s) or organ(s) to be transplanted, placing the obtained osteochondral tissue or other suitable tissue(s) or organ(s) into a semipermeable membrane chamber containing an alginate solution, placing the semipermeable membrane chamber into a culture chamber and adding a calcium solution or a barium solution or both to the culture chamber, thereby encapsulating the osteochondral tissue or other suitable tissue(s) or organ(s) in an alginate gel, injecting culture medium into the alginate gel or the culture chamber or the alginate gel in the culture chamber, and placing the culture chamber into a storage container. In some embodiments, the encapsulated osteochondral tissue or other suitable tissue(s) or organ(s) remains viable for example, for implantation. Additionally or alternatively the encapsulated osteochondral tissue or other suitable tissue(s) or organ(s) remains viable for at least about 2 weeks (such as at least about 4 weeks or at least about 6 weeks) or up to 8 weeks, for example, after the step of obtaining. In various embodiments, the calcium solution comprises, consists essentially of, or further consists of a calcium chloride solution. In various embodiments, the barium solution comprises, consists essentially of, or further consists of a barium chloride solution. In various embodiments, the storage container further comprises a temperature sensor. In various embodiments, the tissue(s) or organ(s) is or are encapsulated in the semipermeable membrane chamber. In various embodiments, the semipermeable membrane has a feature of one or more of a pore size of: at least about 250 pm; or about 0.22 μm to about 1000 μm; and ranges in between, such as about 0.22 μm to about 100 μm or about 100 μm to about 1000 Additionally or alternatively, the semipermeable membrane has a feature of a molecular weight cut-off (MWCO) at about 50 kDa or about 200,000 kDa. In various embodiments, the semipermeable membrane has a mesh opening rate of about 1% to about 50% and ranges or percentages in between, for example about 2%. In various embodiments, the alginate solution is an ultra-purified alginate solution. In various embodiments, the alginate solution comprises or consists essentially of about 0.5% to about 2.0% alginate and ranges or percentages in between, optionally about 1.2% alginate. In various embodiments, the culture medium is complete medium. In various embodiments, the method further includes transporting the storage container to a hospital after storing for a period of time. In various embodiments, the method further comprises maintaining the temperature in the semipermeable membrane, or in the culture chamber, or in the storage container below 10° C., for example, from about 0° C. to about 4° C., or at about 4° C. In various embodiments, the CO₂ level, or the 02 level, or the humidity, or any combination thereof in the semipermeable membrane, or the culture chamber, or the storage container is/are not artificially controlled. In various embodiments, the method further comprises dissolving the alginate gel, for example, prior to implantation, for example, by a method comprising, or consisting essentially or, or consisting of, exposing the encapsulated tissue(s) or organ(s) to a chelating solution. In further embodiments, the chelating solution comprises, consists essentially of, or further consists of a sodium citrate solution or an Ethylenediaminetetraacetic acid (EDTA) solution or a solution comprising or consisting essentially of a sodium citrate and an EDTA. In various embodiments, the method further comprises disassembling the semipermeable membrane, for example, prior to implantation. In various embodiments, the tissue(s) or organ(s) viable, for example, for implantation, reduces or reduce release of inflammatory factor(s), for example, compared to a tissue or an organ stored without an encapsulation in a gel, in a semipermeable membrane, or in both a gel and a semipermeable membrane. In further embodiments, the inflammatory factor(s) comprises, consists essentially of, or further consists of nitric oxide, or one or more of cytokines, or both nitric oxide and one or more of cytokines. In various embodiments, the tissue(s) or organ(s) remains or remain viable for example, for implantation. Additionally or alternatively, the tissue(s) or organ(s) maintains or maintain a cell viability of at least about 60%, or at least about 65%, or at least about 70%, or at least about 75%, or at least about 80%, or at least about 85% or higher of that prior to the encapsulating step. In various embodiments, the method further comprises injecting a buffer, such as a saline, into the alginate gel or the culture chamber or the alginate gel in the culture chamber prior to injecting the culture medium. In further embodiments, the buffer, such as a saline, reduces or removes soluble calcium or barium. In various embodiments, the encapsulated tissue(s) or organ(s) (e.g., cornea) is or are stored for at least about 2 weeks, for example in one aspect, prior to implantation.

In another aspect, the disclosure provides a tissue transporting system. The system includes (for example, comprises, or consists essentially of, or further consists of) a tissue chamber partially or fully formed from a semipermeable membrane and configured to contain tissue to be transported and an alginate solution; a culture chamber having an inlet port and an optional outlet port, the culture chamber being configured to contain the tissue chamber, wherein upon addition of a calcium solution or a barium solution or both through the inlet port, the alginate solution gels and encapsulates the tissue, a transport box or a storage container configured to contain the culture chamber; and an optional temperature sensor disposed in the transport box or the storage container and configured to measure temperature of the culture chamber. In various embodiments, the tissue chamber is enclosed. In various embodiments, the culture chamber is enclosed. In various embodiments, the alginate solution is an ultra-purified alginate (UPAL) solution. In various embodiments, the tissue chamber is an enclosed tissue chamber configured to encapsulate the tissue by the semipermeable membrane. In various embodiments, the semipermeable membrane has/comprises a pore size of at least about 250 pm, for example, about 0.22 μm to about 1000 μm and ranges in between, such as 0.22 μm to about 100 μm, or about 100 μm to about 1000 μm. In various embodiments, the semipermeable membrane has a mesh opening rate of about 1% to about 50% and ranges or percentages in between, for example about 2%. Additionally or alternatively, the semipermeable membrane has a molecular weight cut-off (MWCO) at about 50 kDa or about 200,000 kDa. In various embodiments, the alginate solution comprises, or consists essentially of, or further consists of about 0.5% to about 2.0% alginate and ranges or percentages in between, optionally about 1.2% alginate. In various embodiments, the transport box or the storage container or the culture chamber further comprises a heat insulation component or a cooling component or both a heating insulation component and a cooing component configured to maintain the temperature in the transport box or the storage container or the culture chamber below 10° C., for example, from about 0° C. to about 4° C. and ranges in between, or at about 4° C. In various embodiments, the system does not comprises a sensor or a controller of the CO₂ level, or the 02 level, or the humidity, or any combination thereof in the semipermeable membrane or the culture chamber or the storage container. In various embodiments, the transport box or the storage container is sealed against continuous flow of gases or liquids.

In some embodiments, a tissue culture device, includes (for example, comprises, or consists essentially of, or further consists of) an outer chamber defining an inlet structured to allow inflow of a fluid, and an outlet structured to allow outflow of a fluid therethrough, the outer chamber being enclosed. An inner chamber is disposed at least partially within the outer chamber, the inner chamber having at least one wall formed from a semipermeable membrane, the inner chamber being enclosed and structured to hold a tissue therewithin. The tissue culture device optionally, also includes (for example, further comprises) an enclosed storage container, the outer chamber and thereby, the inner chamber disposed within an internal volume defined by the storage container. In various embodiments, the tissue culture device further comprises a temperature sensor operably coupled to the storage container and configured to measure a temperature within the storage container. In various embodiment, the tissue is encapsulated by an alginate gel. In various embodiments, the fluid comprises, or consists essentially of, or consists of at least one of an alginate solution, a calcium solution, a barium solution, saline, culture media, or storage media. In various embodiments, the semipermeable membrane has a molecular weight cut-off (MWCO) at about 50 kDa or about 200,000 kDa. In various embodiments, the semipermeable membrane has a pore size of at least about 250 pm. In various embodiments, the semipermeable membrane has a pore size in a range of about 0.22 μm to about 100 μm and ranges in between, such as about 100 μm to about 1000 μm, or about 0.22 μm to about 1000 μm. In various embodiments, the semipermeable membrane has a mesh opening rate of about 1% to about 50% and ranges or percentages in between, for example about 2%. In various embodiments, the tissue culture device further comprises at least one of a heat insulation component or a cooling component disposed within the storage container, the at least one of the heat insulation component or the cooling component configured to maintain a temperature within the storage container and/or the outer chamber below 10° C.

In one aspect, a kit as disclosed herein is provided, which comprises or consists essentially of a tissue culture device as disclosed herein and an optional instruction for use. In various embodiments, a kit as disclosed herein further comprises one or more of the following: alginate or any other hydrogel monomer optionally in a solution, a calcium solution, a barium solution, a calcium salt, a barium salt, a solvent, a washing solution, or a culture medium.

### BRIEF DESCRIPTION OF THE DRAWINGS

FIG. 1 is a pictorial diagram showing exemplary storage condition schemas.

FIG. 2 provide a series of graphical diagrams showing the results of chondrocyte zonal viability.

FIGS. 3A-3B provide a pictorial diagram showing representative histological images (FIG. 3A) and a graphical diagram showing histological score results (FIG. 3B).

FIG. 4A is a pictorial diagram showing an exemplary tissue preservation and transport system.

FIGS. 4B-4F are various views of a tissue culture device, according to an embodiment.

FIG. 5 is a pictorial diagram showing exemplary storage conditions and a UPAL Gel coated sample.

FIGS. 6A-6D show normalized percentage of live cell in the stored articular cartilage (FIG. 6A), meniscus (FIG. 6B), patellar tendon (FIG. 6C), and cornea (FIG. 6D). Mean±SE, *=p<0.05, **=p<0.01.

FIG. 7 provides an example of a partial structure of alginate.

### DETAILED DESCRIPTION OF THE DISCLOSURE

The present disclosure is based on the discovery that encapsulation of tissue to be implanted in alginate gel improves the quality and viability of the tissue after long-term term storage, or transportation, or both storage and transportation.

### Definitions

As it would be understood, the section or subsection headings as used herein is for organizational purposes only and are not to be construed as limiting or separating or both limiting and separating the subject matter described.

Before the present compositions and methods are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition; and the more recent editions each thereof available at the time of filing.

As used in this specification and the appended claims, the singular forms “a”, “an”, and “the” include plural references unless the context clearly dictates otherwise. Thus, for example, references to “the method” includes one or more methods, one or more steps, and both one or more methods and one or more steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the compositions and methods corresponding to the scope of each of these phrases. Thus, a composition or method comprising recited elements or steps contemplates particular embodiments in which the composition or method consists essentially of or consists of those elements or steps.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The term “subject” as used herein refers to any individual or patient to which the subject methods are performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, sport animals, pets, etc., and primates (including monkeys, chimpanzees, orangutans and gorillas) are included within the definition of subject.

As used herein, the term “animal” refers to living multi-cellular vertebrate organisms, a category that includes, for example, mammals and birds. The term “mammal” includes both human and non-human mammals.

As used herein, the terms “sample” and “biological sample” refer to any sample suitable for the methods provided by the present disclosure. In one embodiment, the biological sample of the present disclosure is a tissue sample, e.g., a biopsy specimen such as samples from needle biopsy (i.e., biopsy sample).

As used herein, the terms “reduce” and “inhibit” are used together because it is recognized that, in some cases, a decrease can be reduced below the level of detection of a particular assay, for example by about 5%, about 10%, about 15%, about 20%, about 25%, about 30%, about 40%, about 50%, about 60%, about 70%, about 80%, about 85%, about 90%, about 95%, about 99%, or about 100% of the reference level. As such, it may not always be clear whether the expression level or activity is “reduced” below a level of detection of an assay, or is completely “inhibited.” Nevertheless, it will be clearly determinable, following a treatment according to the present methods.

Chondrocyte, as used herein, refers to cells in cartilage. A chondrocyte free of a disease produces and maintains the cartilaginous matrix, which consists mainly of collagen and proteoglycans (PGs). Mesenchymal stem cells are a naturally occurring progenitor of chondrocytes. Other stem cells may derive chondrocytes under suitable conditions, a non-limiting example of which is provided in the Examples.

As used herein, the term “tissue” refers to a cellular organizational level between cells and a complete organ. A tissue comprises, consists essentially of, or further consists of, an ensemble of cells, similar or not, and their extracellular matrix, optionally from the same origin that together carry out a specific function, such as an epithelial tissue, a connective tissue, a muscle tissue, a nervous tissue, an osteochondral tissue, or a fibrocartilage tissue. In some embodiments, a tissue as used herein is an animal tissue, such as a human tissue. In other embodiments, a tissue is a non-human animal tissue.

As used herein, the term “organ” refers to a group of tissues that structurally form a functional unit specialized to perform a particular function, such as a kidney, a liver, a heart, a lung, a bone, or an eye. In some embodiments, an organ as used herein is an animal organ, such as a human organ. In other embodiments, a tissue is a non-human animal organ.

As used herein, an osteochondral tissue comprises, consists essentially of, or further consists of articular cartilage, and a subchondral bone region. In some embodiments, an osteochondral tissue comprises, consists essentially of, or further consists of an osteochondral core. Additionally or alternatively, the osteochondral tissue is obtained from an animal (human or non-human) and optionally trimmed. In further embodiments, the osteochondral tissue has or comprises a bone-to-cartilage-thickness ratio of from about 20:1 to about 1:10. In yet further embodiments, for example, the osteochondral tissue has or comprises a bone-to-cartilage-thickness ratio of about 4:1.

As used herein, an articular cartilage refers to a hyaline cartilage on the articular surfaces of bones, and lies inside the joint cavity of synovial joints, bathed in synovial fluid produced by the synovial membrane, which lines the walls of the cavity.

As used herein, a fibrocartilage tissue refers to a mixture of white fibrous tissue and cartilaginous tissue in various proportions. (e.g., a secondary cartilaginous joint, such as pubic symphysis, annulus fibrosis of intervertebral discs, or manubriosternal joint); glenoid labrum of shoulder joint; acetabular labrum of hip joint; meniscus, including but not limited to medial and lateral menisci of the knee joint; or location where tendons and ligaments attach to bone. In some embodiments, a fibrocartilage tissue as disclosed herein refers to a meniscus, e.g., a medial meniscus, or a lateral meniscus, or both.

As used herein, a tendon refers to a fibrous connective tissue that connects muscle to bone and is capable of withstanding tension. In some embodiments, a tendon as disclosed herein refers to a patellar tendon. Additionally or alternatively, a tendon as disclosed herein refers to an Achilles tendon. In some embodiments, a tendon as used herein is a human tendon. In other embodiments, a tendon as used herein is a non-human animal tendon.

As used herein, a cornea refers to the transparent front part of the eye that covers the iris, pupil, and anterior chamber. In some embodiments, a cornea as used herein refers to a cornea with a vitreous body, which is the clear gel that fills the space between the lens and the retina of the eyeball of humans and other vertebrates. In some embodiments, a cornea as used herein is a human cornea. In other embodiments, a cornea as used herein is a non-human animal cornea.

As used herein, the term “encapsulating” “embedding” or a grammatical variation thereof refers to the orientation or location of a structure or a component so that it is at least in part (for example, at least 99%, or at least 98%, or at least 97%, or at least 96%, or at least 95%, or at least 90%, or at least 85%, or at least 80%, or at least 75%, or at least 70%, or at least 65%, or at least 60%, or at least 55%, or at least 50%, or at least 45%, or at least 40%, or at least 35%, or at least 30%, or at least 25%, or at least 20%, or at least 15%, or at least 10%, or at least 5% of the surface of the structure), in some embodiments completely (for example, about 100% of the surface of the structure), surrounded (directly or indirectly) by one or more other structures or one or more other components. In various embodiments, the term “encapsulating” or a grammatical variation thereof refers to the orientation or location of a structure or a component so that it is completely surrounded by one or more other structures or one or more other components. In some embodiments, the term “encapsulating” refers to being disposed within at least partially or completely. One non-limiting example is a tissue surrounded by an alginate gel, or by an alginate gel and a semipermeable membrane, is referred to herein as an encapsulated tissue.

The term “exposing” or a grammatical variation thereof, refers to contacting, meaning direct or indirect interaction between two or more components. In various embodiments, the term “exposing” refers to encasing or submerging. One non-limiting example as used herein is exposing a tissue (such as a tissue encapsulated in an alginate gel, or a tissue encapsulated in an alginate gel and a semipermeable membrane, or a tissue without any encapsulation) to a culture medium, via a method comprising, consisting essentially of, or consisting of contacting the tissue with the culture medium, for example, encasing or submerging the tissue in the culture medium. A further non-limiting example is exposing a tissue (such as a tissue encapsulated in an alginate gel, or a tissue encapsulated in an alginate gel and a semipermeable membrane, or a tissue without any encapsulation) in a chamber to a culture medium, via a method comprising, consisting essentially of, or consisting of adding the culture medium into the chamber containing the tissue, for example, replacing at least in part (e.g., about 50%, about 60%, about 70%, about 80%, about 90%, or more) or completely (i.e., about 100%) the solution initially present in the chamber, if any.

As used herein, the term preservation or a grammatical variation thereof refers to a storage for a certain time period, such as at least 48 hours, at least 3 days, at least 5 days, at least 1 week, at least 2 weeks, at least 3 weeks, at least at least 4 weeks, at least 5 weeks, at least 6 weeks, at least 7 weeks, at least 8 weeks, at least 9 weeks, at least 10 weeks or longer while maintaining the viability of the stored cells, tissues, organs or other components, for example, having a viability of about 100%, or at least 99%, or at least 98%, or at least 97%, or at least 96%, or at least 95%, or at least 90%, or at least 85%, or at least 80%, or at least 75%, or at least 70%, or at least 65%, or at least 60%, or at least 55%, or at least 50%, or at least 45%, or at least 40%, or at least 35%, or at least 30%, or at least 25%, or at least 20%, or at least 15%, or at least 10%, or at least 5% of the viability prior to the storage. Methods for evaluating the viability of a tissue or an organ is available to one of skill in the art, see, for example, the methods disclosed in the Examples.

As used herein, a culture medium refers to a liquid substance used for culturing a cell or a tissue or an organ, for example, to support the cell growth or, to keep the cell alive, or to maintain the tissue viability. In some embodiments, a culture medium comprises, consists essentially of, or further consists of: one or more of amino acid(s), or one or more of carbohydrate(s) as an energy source, or one or more of trace elements, or one or more of vitamins, or one or more of salts, or one or more of possible additional components (e.g., to affect cell growth and/or productivity and/or product quality), or any combination thereof. In some embodiments, the culture medium comprises, consists essentially of, or further consists of Dulbecco's Modified Essential Medium (DMEM), or Ham's F-12 Medium (F12), or Roswell Park Memorial Institute (RPMI) 1640 medium, or any combination thereof. In further embodiments, the culture medium comprises, consists essentially of, or further consists of DMEM/F12 (a 1:1 mixture of DMEM and F12),

In some embodiments, a complete medium refers to a culture medium further comprising one or more of additional components that may contribute to cell growth, such as one or more of growth factors, one or more of hormones, one or more of proteins, one or more of serum, one or more of serum substitute. In further embodiments, a complete medium refers to a culture medium further comprising a serum, such as fetal bovine serum (FBS). Additionally or alternatively, a complete medium further comprises about 1% to about 20%, (such as about 1%, or about 2%, or about 3%, or about 4%, or about 5%, or about 6%, or about 7%, or about 8%, or about 9%, or about 10%, or about 11%, or about 12%, or about 13%, or about 15%, or about 20%) serum.

As used herein, a semipermeable membrane, which is referred to as a dialysis membrane, means a type of biological or synthetic, polymeric membrane that allows certain particles (such as molecules or ions) to pass through it by osmosis—or occasionally by more specialized processes of facilitated diffusion, passive transport or active transport, thereby allowing particles to pass through by size or molecular weight.

For example, semipermeable membranes come with various pore sizes, permitting particles smaller than their pore sizes to pass. In some embodiments, the pore size of a semipermeable membrane is provided by the diameter of the membrane pores. In further embodiments, the pore size of a semipermeable membrane is provided by the averaged diameter of the membrane pores. In some embodiments, a semipermeable membrane as disclosed herein has a pore size of at least about 250 picometer (“pm” as provided herein), at least about 300 pm, at least about 500 pm, at least about 750 pm, at least about 1 nm, at least about 20 nm, at least about 50 nm, at least about 100 nm, at least about 150 nm, at least about 200 nm, at least about 300 nm, at least about 400 nm, at least about 500 nm, at least about 600 nm, at least about 700 nm, at least about 800 nm, at least about 900 nm, at least about 1 μm, at least about 20 μm, at least about 50 μm, at least about 100 μm, at least about 150 μm, at least about 200 μm, at least about 300 μm, at least about 400 μm, at least about 500 μm, at least about 600 μm, at least about 700 μm, at least about 800 μm, at least about 900 μm, at least about 1000 μm, at least about 1100 μm, at least about 1200 μm, at least about 1300 μm, at least about 1400 μm, at least about 1500 μm, at least about 1600 μm, at least about 1700 μm, at least about 1800 μm, at least about 1900 μm, at least about 2000 μm, at least about 2100 μm, at least about 2200 μm, at least about 2300 μm, at least about 2400 μm, at least about 2500 μm, at least about 2600 μm, at least about 2700 μm, at least about 2800 μm, at least about 3900 μm, at least about 3000 μm, at least about 4000 μm, at least about 5000 μm, at least about 6000 μm, at least about 7000 μm, at least about 8000 μm, at least about 9000 μm, at least about 10 mm, or larger and ranges in between. Additionally or alternatively, a semipermeable membrane as disclosed herein has a pore size of up to about 300 pm, up to about 500 pm, up to about 750 pm, up to about 1 nm, up to about 20 nm, up to about 50 nm, up to about 100 nm, up to about 150 nm, up to about 200 nm, up to about 300 nm, up to about 400 nm, up to about 500 nm, up to about 600 nm, up to about 700 nm, up to about 800 nm, up to about 900 nm, up to about 1 μm, up to about 20 μm, up to about 50 μm, up to about 100 μm, up to about 150 μm, up to about 200 μm, up to about 300 μm, up to about 400 μm, up to about 500 μm, up to about 600 μm, up to about 700 μm, up to about 800 μm, up to about 900 μm, up to about 1000 μm, up to about 1100 μm, up to about 1200 μm, up to about 1300 μm, up to about 1400 μm, up to about 1500 μm, up to about 1600 μm, up to about 1700 μm, up to about 1800 μm, up to about 1900 μm, up to about 2000 μm, up to about 2100 μm, up to about 2200 μm, up to about 2300 μm, up to about 2400 μm, up to about 2500 μm, up to about 2600 μm, up to about 2700 μm, up to about 2800 μm, up to about 3900 μm, up to about 3000 μm, up to about 4000 μm, up to about 5000 μm, up to about 6000 μm, up to about 7000 μm, up to about 8000 μm, up to about 9000 μm, up to about 10 mm, or larger. In some embodiments, a semipermeable membrane as disclosed herein has a pore size of about 0.22 μm to about 1000 μm, and ranges or sizes in between, such as about 0.22 μm to about 100 μm and ranges in between, or about 100 μm to about 1000 μm and ranges in between. In some embodiments, a semipermeable membrane as disclosed herein has a pore size of about 100 μm to about 1000 μm and ranges in between, such as 100 μm to about 200 μm, 100 μm to about 300 μm, 100 μm to about 400 μm, 100 μm to about 500 μm, 100 μm to about 600 μm, 100 μm to about 700 μm, 100 μm to about 800 μm, or about 100 μm to about 900 μm. In various embodiments, a pore size of the semipermeable membrane as disclosed herein is selected by one of skill in the art so that one or more of the component(s) of a culture medium (such as a complete medium) can pass the membrane but not one or more of: a cell, a tissue (for example an encapsulated tissue), or an alginate gel.

In some embodiments, a molecular weight cut-off (MWCO) is used to describe the permeability of a semipermeable membrane, referring to the lowest molecular weight (such as in Daltons (Da) or kilodaltons (kDa)) at which greater than 90% of a particle with the molecular weight is retained by the membrane. In various embodiments, an MWCO of a semipermeable membrane is selected or determined upon selection of the pore size. In other embodiments, a pore size of a semipermeable membrane is selected or determined upon selection of the MWCO. In various embodiments, an MWCO is selected based on a solution as used herein and its concentration, such as a calcium solution, a barium solution or a chelating solution. In various embodiments, an MWCO of the semipermeable membrane as disclosed herein is selected by one of skill in the art so that one or more of the component(s) of a culture medium (such as a complete medium) can pass the membrane but not one or more of: a cell, a tissue (for example an encapsulated tissue), or an alginate gel. In some embodiments, MWCO of a semipermeable membrane as disclosed herein is about 1 kDa to about 10,000,000 kDa and ranges in between, for example about 1 kDa to about 2,000.00 kDa. In some embodiments, MWCO of a semipermeable membrane as disclosed herein is at least about 1 kDa, at least about 10 kDa, at least about 50 kDa, at least about 100 kDa, at least about 200 kDa, at least about 300 kDa, at least about 400 kDa, at least 500 kDa, at least about 600 kDa, at least about 700 kDa, at least about 800 kDa, at least about 900 kDa, at least about 1,000 kDa, at least about 2,000 kDa, at least about 3,000 kDa, at least about 4,000 kDa, at least about 5,000 kDa, at least about 6,000 kDa, at least about 7,000 kDa, at least about 8,000 kDa, at least about 9,000 kDa, at least about 10,000 kDa, at least about 20,000 kDa, at least about 30,000 kDa, at least about 40,000 kDa, at least about 50,000 kDa, at least about 60,000 kDa, at least about 70,000 kDa, at least about 80,000 kDa, at least about 90,000 kDa, at least about 100,000 kDa, at least about 200,000 kDa, at least about 300,000 kDa, at least about 400,000 kDa, at least about 500,000 kDa, at least about 600,000 kDa, at least about 700,000 kDa, at least about 800,000 kDa, at least about 900,000 kDa, at least about 1,000,000 kDa, at least about 2,000,000 kDa, at least about 3,000,000 kDa, at least about 4,000,000 kDa, at least about 5,000,000 kDa, at least about 6,000,000 kDa, at least about 7,000,000 kDa, at least about 8,000,000 kDa, or at least about 9,000,000 kDa. Additionally or alternatively, MWCO of a semipermeable membrane as disclosed herein is up to about 50 kDa, up to about 100 kDa, up to about 200 kDa, up to about 300 kDa, up to about 400 kDa, up to 500 kDa, up to about 600 kDa, up to about 700 kDa, up to about 800 kDa, up to about 900 kDa, up to about 1,000 kDa, up to about 2,000 kDa, up to about 3,000 kDa, up to about 4,000 kDa, up to about 5,000 kDa, up to about 6,000 kDa, up to about 7,000 kDa, up to about 8,000 kDa, up to about 9,000 kDa, up to about 10,000 kDa, up to about 20,000 kDa, up to about 30,000 kDa, up to about 40,000 kDa, up to about 50,000 kDa, up to about 60,000 kDa, up to about 70,000 kDa, up to about 80,000 kDa, up to about 90,000 kDa, up to about 100,000 kDa, up to about 200,000 kDa, up to about 300,000 kDa, up to about 400,000 kDa, up to about 500,000 kDa, up to about 600,000 kDa, up to about 700,000 kDa, up to about 800,000 kDa, up to about 900,000 kDa, up to about 1,000,000 kDa, up to about 2,000,000 kDa, up to about 3,000,000 kDa, up to about 4,000,000 kDa, up to about 5,000,000 kDa, up to about 6,000,000 kDa, up to about 7,000,000 kDa, up to about 8,000,000 kDa, up to about 9,000,000 kDa, up to about 10,000,000 kDa or larger. In some embodiments, MWCO of a semipermeable membrane as disclosed herein is about 10 kDa, or about 20 kDa, or about 30 kDa, or about 40 kDa, or about 50 kDa, or about 60 kDa, or about 70 kDa, or about 80 kDa, or about 90 kDa, or about 100 kDa, or about 200,000 kDa.

In some embodiments, the semipermeable membranes may be partially or fully semipermeable. For example, a partially semipermeable membrane may selectively allow transport of certain fluids therethrough. In contrast, a fully semipermeable membrane may allow unrestricted transport of fluid therethrough, but prevent transport of the alginate gel and the tissue therethrough. Such fully semipermeable membranes may comprises, consists essentially of, or alternatively consists of a mesh, for example, a medical grade mesh. Accordingly, in some embodiments, a mesh opening size refers to a pore size of the mesh or an average thereof, which may be used interchangeably with a membrane pore size.

In some embodiments, a semipermeable membrane has an opening rate (which is also referred to herein as a mesh opening rate) of about 1% to about 50% and ranges or percentages in between. In some embodiments, a semipermeable membrane has a mesh opening rate of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 15%, at least about 20%, at least about 25%, at least about 30%, at least about 35%, at least about 40%, or at least about 45%. Additionally or alternatively, a semipermeable membrane has an opening rate of less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95%. In some embodiments, a semipermeable membrane has a mesh opening rate of about 1%, about 2%, about 3%, about 4%, about 5%, about 6%, about 7%, about 8%, about 9%, about 10%, about 11%, about 15%, about 20%, about 25%, about 30%, about 35%, about 40%, about 45%, or about 50% s.

In some embodiments, an opening rate of a membrane refers to a percentage or a ratio of the opening area over the entire surface of the membrane.

As used herein, a percentage (%) is used to indicate a concentration of a component in a mixture (solid or liquid). In some embodiments, a percentage (%) refers to a mass ratio or weight ratio. In other embodiments, a percentage (%) refers to a volume ratio. In yet other embodiments, a percentage (%) refers to a molar ratio. In some embodiments, x % of a component in a solution refers to x gram of the component in 100 mL of the solution.

As used herein, a chelating solution refers to a solution comprising a chelating agent, such as a calcium chelating agent. Non-limiting examples of the chelating agent include edetic acid, citric acid, edetate disodium anhydrous, edetate calcium disodium anhydrous, Ethylenediaminetetraacetic acid (EDTA), or ethylene glycol-bis(β-aminoethyl ether)-N,N,N′,N′-tetraacetic acid (EGTA). In some embodiments, the chelating agent is sodium citrate, or Ethylenediaminetetraacetic acid (EDTA). In some embodiments, the chelating solution is a sodium citrate solution or an EDTA solution, or a mixture thereof.

In some embodiments, the solution is an aqueous solution, and the solution may contain additional ingredients such as saline.

An inflammatory response, as used herein interchangeably with inflammation, refers to an immune response that occurs when tissues are injured by bacteria, trauma, toxins, heat, or any other cause, such as ex vivo preservation. The tissue releases inflammatory factors during the inflammation response, such as histamine, bradykinin, serotonin, a cytokine, nitric oxide. As used herein, cytokines, such as interleukin (IL)-1, IL-1β, IL-6 and tumor necrosis factor-α (TNFα), are produced by an immune cell (such as activated macrophage), a tissue or an organ, or a chondrocyte.

As used herein, the term “allograft” refers to a tissue graft from a donor of the same species as the recipient, but is not genetically identical. Allograft tissue can be removed from a donor by techniques known in the art. For instance, general aseptic surgical methods or other physical intervention of an allograft may include but are not limited to excision, resection, amputation, transplantation, microsurgery, general surgery, laser surgery, robotic surgery, or autopsy, among others.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

### MODES FOR CARRYING OUT THE DISCLOSURE

Tissue or allograft sources may be cells, tissues, or organs from all types of organisms, including, but not limited to human, porcine, ovine, bovine, canine, equine, and others. In one embodiment, the source of the tissue or allograft is human. Potential tissue or allograft sources may include, but are not limited to, tissues of the eye, brain, heart, kidney, liver, intestine, bone, cartilage, skin, lung, thyroid, stomach, ligaments, tendons, or any other tissue or cell source that may require transplantation. In various embodiments, the allograft or tissue may include bone or cartilage or meniscus tissue of the spine, scapula, humerus, radius, ulna, pelvis, femur, tibia, fibula, patella, talus, phalanges, or temporomandibular joint, or any combination thereof. In various embodiments, the allograft or tissue may be osteochondral tissue. Although the description herein may refer to allograft tissue, one of skill in the art appreciates that other tissues find use in the methods described herein.

In some embodiments, a tissue encapsulated by a gel, a semipermeable membrane, or both a gel and a semipermeable membrane is longer than about 1 millimeter (mm) in every dimension of the tissue, for example at least about 2 mm, at least about 3 mm, at least about 4 mm, at least about 5 mm, at least about 6 mm, at least about 7 mm, at least about 8 mm, at least about 9 mm, at least about 1 centimeter (cm), or longer. Additionally or alternatively, a tissue encapsulated by a gel, a semipermeable membrane, or both a gel and a semipermeable membrane is no longer than about 20 cm in every dimension of the tissue, for example, no longer than about 15 cm, no longer than about 10 cm, no longer than about 5 cm, no longer than about 4 cm, no longer than about 3 cm, no longer than about 2 cm, no longer than about 1 cm, or shorter.

Osteochondral allografts (OCAs) are currently preserved at 4° C. and used within 28 days of donor harvest. The window of opportunity for implantation is limited to 14 days due to a two-week disease testing protocol, severely limiting availability to potential recipients. The present study was performed to find improved storage conditions that increases the shelf life of OCAs and the availability of their clinical usage with an improved clinical outcome.

Ultra-purified alginate (UPAL) is highly purified with reduced endotoxicity (<1/10,000 compared to conventional alginate). In some embodiments, an UPAL is purified from a conventional alginate by a method comprising, consisting essentially of, or consisting of adding an acid to a conventional alginate to precipitate the alginate and isolating the insoluble alginate. See, for example, Igarashi, et al., Cartilage 2012:70. As a scaffold material, UPAL has been shown to enhance the repair of osteochondral defects in animal models (Igarashi, et al., Cartilage 2012:70; and Baba, et al., Am J Sports Med. 2018; 1970) and, as an intra-articular injectable material, to delay the progression of osteoarthritis (Tsukuda, et al., J Biomed Mater Res A. 2015:3441). The similarity of alginate to the articular cartilage matrix and its preferred use for 3D culture in the form of alginate beads and as an embedding material (Chiba, et al., Spine J. 2007:694), suggests that UPAL can be expected to act as an optimum storage medium for preventing OCAs from degrading. It was therefore hypothesized that the modification of current storage conditions using UPAL improves the quality of OCAs after long-term storage. The specific objective of this study is to evaluate the effects of UPAL on cell viability and cartilage matrix status after 4-weeks storage of OCAs obtained from pig knees.

Alginic acid, also called algin, is a polysaccharide distributed widely in the cell walls of brown algae that is hydrophilic and forms a viscous gum when hydrated. With metals such as sodium and calcium, its salts are known as alginates. Sugars in alginate consist of guluronate (G), mannuronate (M) or guluronate-mannuronate blocks, and the proportion of the different sugars determines how strong a gel is formed. In some embodiments, the M/G ratio of the alginic acids and/or salts thereof used is usually in a range selected from of about 0.2 to about 4.0, in a range of about 0.4 to about 3.0, and in a range of about 0.5 to about 3.0, and ranges in between. Polymer substance derived from a natural product is generally an aggregate of molecules having various molecular weights, and thus is measured as a molecular weight distribution having a certain range. The molecular weight of alginic acid can be measured by a standard method of calculating by SEC-MALS (Size Exclusion Chromatography with Multiple Angle Laser Light Scattering Detection) (ASTM F2605-16 (2016), published by ASTM International). In some embodiments, the molecular weight of alginic acids and/or salts thereof used is usually in a range selected of about 10,000 to about 2,000,000, in a range of about 15,000 to about 1,500,000, in a range of about 20,000 to about 1,000,000, or in a range of about 25,000 to about 500,000, and ranges in between, as measured by SEC-MALS method.

It is well known that high chondrocyte viability results in good in vivo performance (Pallante, et al., Am J Sports Med. 2012:1814). The encapsulation of OCAs by UPAL gel resulted in a significantly higher chondrocyte viability in the superficial zone compared to the other groups. The viability in the middle zone and total thickness in the UPAL gel group also showed a tendency to be higher than that in the other groups. These results are concordant with the results that the superficial zone was vulnerable, and that cell viability of the superficial zone was susceptible to its storage condition (Pallante, et al., Am J Sports Med. 2009:24S). UPAL gel encapsulation showed no deformation of its structure during the storage period. The maintenance of cell viability and matrix content, as well as polyglycan (PG) synthesis, in the alginate gel is also supported by the result of the low concentration of total nitric oxide (NO) in the storage medium. Embedment in alginate gel may also provide tissues some protection from mechanical damage during tissue transport to the clinical site. The use of UPAL gel encapsulation may be effective for improving the quality of OCAs after 28 days of storage. Thus, the use of UPAL gel during storage has potential to improve clinical outcomes after osteochondral allografting with minimized cell death during the storage period.

As exemplified in the examples disclosed herein, embedding (i.e., encapsulating) an osteochondral allograft (OCA) in UPAL gel improves cell viability and matrix composition after 28 days storage or after 6 weeks storage. Matrix synthesis is preserved compared to the OCA stored in other control methods. Further, the cell viability with alginate was higher than the one without alginate (a control culture). The storage of grafted tissues with UPAL gel showed significantly higher cell viability than that of tissues stored in media alone. The UPAL membrane group, which has tissues embedded in UPAL gel and a semipermeable membrane, showed cell viability higher than those of all other testing conditions. Without wishing to be bound by the theory, conditions in the UPAL membrane group provides excellent access to nutrients through the UPAL gel and membrane and also provides physical protection during the storage period. In addition, embedment in alginate gel alone provides the tissue some protection from mechanical damage during the storage period. At the end of the storage period, alginate gel containing tissues is removed from the apparatus or the system by replacing the media with a chelating solution. Any remaining alginate gel around the tissues is dissolved by exposure to a chelating solution, such as EDTA or sodium citrate; which may be done at the operation room just before transplantation. In addition, embedment in alginate gel provides tissue some protection from mechanical damage during tissue transport to the clinical site.

There are a few methods to store cartilage tissues available, but the method, apparatus or system as disclosed herein provides advantage(s) over other methods, for example, during transportation. Such advantages, for example over the liquid storage system, include, but are not limited to, the tissue preservation condition is improved by embedding in alginate gel; the tissue is protected from a shock, vibration or shaking during transporting tissues to hospitals, thereby the tissue damage by transporting being prevented; cell viability is improved and inflammatory factors, such as cytokines and nitric oxide, is suppressed; and one enclosed system covers the storage and transport stage. Therefore, the preservation of allograft tissues embedded in alginate gel and an optional semipermeable membrane improves the maintenance of cells in tissues. In some embodiments, the semipermeable membrane may be partially or fully semipermeable. For example, a partially semipermeable membrane may selectively allow transport of certain fluids therethrough. In contrast, a fully semipermeable membrane may allow unrestricted transport of fluid therethrough, but prevent transport of the alginate gel and the tissue therethrough. Such fully semipermeable membranes may include (such as comprises, consists essentially of, or alternatively consists of) a mesh, for example, a medical grade mesh. Such modification of storage conditions improves quality of a tissue as disclosed herein, such as an osteochondral allograft.

Accordingly, in one aspect, the disclosure provides methods, systems or apparatuses of tissue preservation. The method includes encapsulating the tissue in UPAL gel and storing the encapsulated tissue for, for example, from about 24 hours to about 8 weeks prior to implantation. In various embodiments, the tissue is osteochondral tissue. In various embodiment, the tissue is a tissue as disclosed herein other than an osteochondral tissue.

An unexpected benefit of the present procedure as disclosed herein in any aspect or embodiments is that tissue samples can be maintained viable and sterile for an extended period of time relative to methods of the prior art. For instance, typically in the prior art, upon removal of an allograft from a donor, the tissue was stored on ice or at around 4° C. Tissues prepared according to this method tended to remain suitably viable for around 21-28 days. However, the procedure described herein provides for a surprising and unexpected increase in viability of allograft tissue. Tissues prepared and stored according to the procedure described herein remain viable for an extended period of time relative to storage at 4° C. By an extended period is meant at least between about 7-100 days, at least between about 20-80 days, or at least between about 29-70, 40-70, 50-70 or 60-70 days. In various embodiments, the encapsulated tissue is stored for about 1 day, 2 days, 7 days, 14 days, 28 days, 4 weeks, 6 weeks, or 8 weeks prior to use, such as implantation. In various embodiments, at least about 40% of the cells of the tissue remain viable after the storing, as compared to the viability of the cells of the tissue at day 0.

In another aspect, the disclosure provides a method for storing osteochondral tissue or other tissue(s) or organ(s) as disclosed herein, for example prior to implantation. The method includes (for example, comprises, consists essentially of, or alternatively consists of) osteochondral tissue or other tissue(s) or organ(s) to be implanted, placing the obtained osteochondral tissue or other tissue(s) or organ(s) into a semipermeable membrane chamber containing an alginate solution, placing the semipermeable membrane chamber into a culture chamber and adding a calcium solution or a barium solution or both to the culture chamber, thereby encapsulating the osteochondral tissue or the other tissue(s) or organ(s) in an alginate gel, injecting culture media into the culture chamber, and optionally placing the culture chamber into a storage container. In various embodiments, the encapsulated osteochondral tissue remains viable for implantation for up to 8 weeks after the step of obtaining.

Provided herein are methods, materials, formulations, systems or apparatuses pertaining to the preservation of allograft tissues embedded in alginate gel and an optional semipermeable membrane which improves the maintenance of cells in tissues. In some embodiments, the methods, materials, formulations, systems or apparatuses also provide tissues some protection from mechanical damage during transport to the clinical site.

In one aspect, the disclosure provides a method for osteochondral tissue preservation. In another aspect, provided is a method for preservation of other tissue(s) or organ(s) as disclosed herein. In some embodiments, the tissue(s) or organ(s) is selected from an osteochondral tissue (such as an osteochondral core trimmed to have a bone-to-cartilage-thickness ratio of from about 20:1 to about 1:10 and ranges in between, for example, about 4:1), an articular cartilage free of any subchondral bone, a fibrocartilage tissue (such as a meniscus, e.g., a medial meniscus or a lateral meniscus or both), a tendon (such as a patellar tendon or an Achilles tendon), a cornea, or a cornea with a vitreous body.

In some embodiments, the method includes (for example, comprises, or consists essentially of, or further consists of) encapsulating the osteochondral tissue or other suitable tissue(s) or organ(s) in an alginate gel and exposing the gel-encapsulated osteochondral tissue or other suitable tissue(s) or organ(s) to a culture medium. In some embodiments, the gel-encapsulated tissue is in the culture medium for a time period selected from about 24 hours to about 8 weeks and ranges in between, for about 28 days, for at least about 6 weeks, for example, for about 24 hours to 8 weeks prior to implantation. Additionally or alternatively, the method includes (for example, comprises, or consists essentially of, or further consists of) encapsulating the osteochondral tissue or other suitable tissue(s) or organ(s) in an alginate gel and exposing the encapsulated osteochondral tissue or other suitable tissue(s) or organ(s) to a culture medium for at least about 2 weeks, such as at least about 4 weeks, at least about 5 weeks, at least about 6 weeks, at least about 7 weeks, at least about 8 weeks, at least about 9 weeks, at least about 10 weeks, or longer. In some embodiments, the method includes (for example, comprises, or consists essentially of, or further consists of) encapsulating the osteochondral tissue or other suitable tissue(s) or organ(s) in an alginate gel and exposing the encapsulated osteochondral tissue or other suitable tissue(s) or organ(s) to a culture medium for about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 5 weeks to about 6 weeks, about 5 weeks to about 7 weeks, about 5 weeks to about 8 weeks, about 5 weeks to about 9 weeks, about 5 weeks to about 10 weeks, about 6 weeks to about 7 weeks, about 6 weeks to about 8 weeks, about 6 weeks to about 9 weeks, about 6 weeks to about 10 weeks, or ranges in between of each.

In various embodiments, the method further comprises encapsulating the tissue(s) or organ(s) in a semipermeable membrane. In various embodiments, the semipermeable membrane has a pore size of at least about 250 pm, such as about 0.22 μm to about 100 μm and ranges in between, about 0.22 μm to about 1000 μm and ranges in between, or about 100 μm to about 1000 μm and ranges in between. Additionally or alternatively, the semipermeable membrane has a molecular weight cut-off (MWCO) at about 50 kDa or about 200,000 kDa. In some embodiments, the semipermeable membrane has a mesh opening rate of about 1% to about 50% and ranges or percentages in between, for example about 2%. In various embodiments, the tissue is encapsulated in the gel prior to, during, or after being encapsulated in the semipermeable membrane.

In various embodiments, the alginate gel is a purified gel, or an ultra-purified alginate gel (UPAL). In various embodiments, the alginate gel comprises or consists essentially of about 0.5% to about 2.0% alginate and ranges or percentages in between, for example, about 1.2% alginate. In various embodiments, the UPAL is present in the gel at a concentration of about 0.5% to about 2.0% and ranges or percentages in between, for example, about 1.2%.

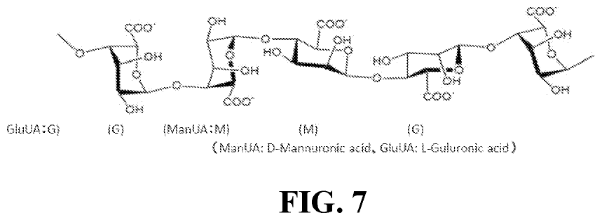

Alginate is a natural polysaccharide that comprises 30 to 60% brown algae (kelp, etc.). Alginate is an aggregate of heteropolymer, comprising, consisting essentially of, or consisting of D-mannuronic acid (M) and Lguluronic acid (G). The carboxyl group, composed of M and G, has high ion-exchange capacity and easily links to various cations. See, an example of a partial structure of alginate in FIG. 7. The physical properties of alginate change in various ways according to the different kinds of cation. Especially, the cross-linking of alginate by calcium ion forms water-insoluble hydrated gel. Alginates of mean molecular weight in the range 10⁴ to 10⁶ Da are available. The higher the molecular weight of alginate, the higher the viscosity in solution. Sodium alginate is a white freeze-dry powder and water-soluble, while Calcium alginate is a white powder and water-insoluble. Purification process of alginate from raw materials comprises, consists essentially of, or alternatively consists of: Dry and crush; wash and swelling; extraction; filtration; solidification and precipitation; and alginate (wet)—drying. A wide ranges of applications have been identified, such as in food (for example, as thickeners, gelling agents, emulsifiers, noodle quality improvers, pet food, aquaculture fish feed, binders and thickeners, etc.), cosmetics, textile printing paste, etc. as well as in pharmaceutical filed, for example as a disintegrator for tablets, or a protective material for stomach wall. Alginate is also the main component of impression material used in dentistry. Alginate also has superior chondrogenic potential.

An ultra pure alginate (UPAL) was developed. Conventional alginate has high endotoxin level and cannot be used for clinical other than oral administration or wound dressing. High endotoxin level results in inflammatory response in vivo. This adverse effect disturbs the clinical application of alginate-based materials. Mochida has developed highly purified alginate with very low level of endotoxin, called UPAL. UPAL eliminates inflammatory response in vivo caused by endotoxin and enabled clinical application of alginate based material. For example, UPAL gel enhances the cellular proliferation of bone marrow stromal cells (BMSCs). See, e.g., Igarashi, Iwasaki, et al. JBMR A, 2010. Various tests revealed that UPAL gel promotes the regeneration of hyaline like cartilage. Clinical study is conducted with applicants who wants to use sodium alginate in the fields of regeneration medicine or cell medicine.

In various embodiments, the alginate (ALG) as used herein is of various molecular weights, such as 10⁴ to 10⁶ Da and ranges and numbers in between. The higher the molecular weight of alginate is, the harder and more elastic of the alginate gel forms. It would be ready for one of skill in the art to select a suitable molecular weight alginate in practicing a method as disclosed herein.

In various embodiments, the culture medium is complete medium.

In various embodiments, the encapsulated osteochondral tissue or other suitable tissue(s) or organ(s) is or are stored for about 28 days (about 4 weeks) prior to implantation or for about 6 weeks, such as prior to implantation. In some embodiments, the encapsulated tissue(s) or organ(s) is or are stored for at least about 2 weeks, such as at least about 4 weeks, at least about 5 weeks, at least about 6 weeks, at least about 7 weeks, at least about 8 weeks, at least about 9 weeks, at least about 10 weeks, or longer, for example, prior to implantation. In some embodiments, the encapsulated tissue(s) or organ(s) is or are stored for about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 5 weeks to about 6 weeks, about 5 weeks to about 7 weeks, about 5 weeks to about 8 weeks, about 5 weeks to about 9 weeks, about 5 weeks to about 10 weeks, about 6 weeks to about 7 weeks, about 6 weeks to about 8 weeks, about 6 weeks to about 9 weeks, about 6 weeks to about 10 weeks, or ranges in between of each, for example, prior to implantation. In various embodiments, the encapsulated cornea is stored for at least about 2 weeks prior to implantation.

In various embodiments, the culture medium is not replenished after exposing the gel-encapsulated tissue to the culture medium. In various embodiments, the culture medium is only replenished no more than once, or no more than twice, or no more than three times, or no more than four times, or no more than five times, after exposing the gel-encapsulated tissue to the culture medium.

In various embodiments, the encapsulated tissue(s) or organ(s) is or are stored at a temperature lower than 10° C., for example, from about 0° C. to about 4° C. and ranges in between, or at about 4° C.

In various embodiments, the encapsulated tissue(s) or organ(s) is or are not stored with an artificially controlled CO₂ level, or an artificially controlled 02 level, or an artificially controlled humidity, or any combination thereof.

In various embodiments, the method further comprises dissolving the alginate gel after the exposure with the culture medium and optionally prior to implantation, for example, by a method comprising, or consisting essentially or, or consisting of, exposing the encapsulated tissue(s) or organ(s) to a chelating solution. In further embodiments, the chelating solution comprises, consists essentially of, or further consists of a sodium citrate solution or an Ethylenediaminetetraacetic acid (EDTA) solution or a solution comprising both sodium citrate and EDTA.

In various embodiments, the method further comprises disassembling the semipermeable membrane after the exposure with the culture medium and prior to implantation.

In various embodiments, the encapsulated tissue(s) or organ(s) reduces or reduce release of inflammatory factor(s), for example, compared to a tissue or organ stored without the encapsulation in an alginate gel, or in a semipermeable membrane, or both in an alginate gel and in a semipermeable membrane. In further embodiments, the inflammatory factor(s) comprises, consists essentially of, or further consists of one or more of cytokines, or nitric oxide, or both nitric oxide and one or more of cytokines.

In various embodiments, the encapsulated tissue(s) or organ(s) prior to the implantation maintains or maintain a cell viability of at least about 60%, or at least about 65%, or at least about 70%, or at least about 75%, or at least about 80%, or at least about 85%, or at least 90%, or at least 95% or higher of that prior to the encapsulating step or after the obtaining step (i.e., a step comprising, consisting essentially of, or consisting of obtaining the tissue(s) or organ(s), such as from a subject).

In various embodiments, prior to exposing to the culture medium, the encapsulated tissue(s) or organ(s) is or are washed with a buffer, such as a saline or a culture medium.

In one aspect, the disclosure provides a method for storing osteochondral tissue. In another aspect, provided is a method for storing tissue(s) or organ(s) as disclosed herein. In some embodiments, the tissue(s) or organ(s) is selected from an osteochondral tissue (such as an osteochondral core trimmed to have a bone-to-cartilage-thickness ratio of from about 20:1 to about 1:10, for example, about 4:1), an articular cartilage free of any subchondral bone, a fibrocartilage tissue (such as a meniscus, e.g., a medial meniscus or a lateral meniscus or both a medial meniscus and a lateral meniscus), a tendon (such as a patellar tendon or an Achilles tendon), a cornea, or a cornea with a vitreous body prior to implantation.

In some embodiments, the method includes (for example, comprises, or consists essentially of, or further consists of) optionally obtaining osteochondral tissue or other suitable tissue(s) or organ(s) to be transplanted, placing the obtained osteochondral tissue or other suitable tissue(s) or organ(s) into a semipermeable membrane chamber containing an alginate solution, placing the semipermeable membrane chamber into a culture chamber and adding a calcium solution or a barium solution or both to the culture chamber, thereby encapsulating the osteochondral tissue or other suitable tissue(s) or organ(s) in an alginate gel, injecting culture medium into the alginate gel or the culture chamber or the alginate gel in the culture chamber, and optionally placing the culture chamber into a storage container.

In some embodiments, the encapsulated osteochondral tissue or other suitable tissue(s) or organ(s) remains or remain viable for implantation for at least about 2 weeks (such as at least about 4 weeks or at least about 6 weeks). In some embodiments, the encapsulated osteochondral tissue or other suitable tissue(s) or organ(s) remains or remain viable for implantation for up to 8 weeks after the step of obtaining. In some embodiments, the encapsulated osteochondral tissue or other suitable tissue(s) or organ(s) remains or remain viable for implantation for at least about 2 weeks, such as at least about 4 weeks, at least about 5 weeks, at least about 6 weeks, at least about 7 weeks, at least about 8 weeks, at least about 9 weeks, at least about 10 weeks, or longer. In some embodiments, the encapsulated osteochondral tissue or other suitable tissue(s) or organ(s) remains or remain viable for implantation for about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 5 weeks to about 6 weeks, about 5 weeks to about 7 weeks, about 5 weeks to about 8 weeks, about 5 weeks to about 9 weeks, about 5 weeks to about 10 weeks, about 6 weeks to about 7 weeks, about 6 weeks to about 8 weeks, about 6 weeks to about 9 weeks, or about 6 weeks to about 10 weeks. In various embodiments, the encapsulated cornea remains viable for implantation for at least about 2 weeks.

In various embodiments, the calcium solution comprises, consists essentially of, or further consists of a calcium chloride solution. In some embodiments, the calcium solution as used herein may be substituted with a barium solution, for example a barium chloride solution. In further embodiments, the calcium solution as used herein may be substitute with a solution comprising or consisting essentially of a calcium salt and a barium salt, such as calcium chloride and barium chloride.

In various embodiments, the storage container further comprises a temperature sensor.

In various embodiments, the tissue(s) or organ(s) is or are encapsulated in the semipermeable membrane chamber. In various embodiments, the semipermeable membrane has a pore size of at least about 250 pm, such as about 0.22 μm to about 100 μm and ranges in between, about 0.22 μm to about 1000 μm and ranges in between, or about 100 μm to about 1000 μm and ranges in between. Additionally or alternatively, the semipermeable membrane has a molecular weight cut-off (MWCO) at about 50 kDa or about 200,000 kDa. In some embodiments, the semipermeable membrane has/comprises a mesh opening rate of about 1% to about 50% and ranges or percentages in between, for example about 2%.

In various embodiments, the alginate solution is an ultra-purified alginate solution. In various embodiments, the alginate solution comprises or consists essentially of about 0.5% to about 2.0% alginate and ranges or percentages in between, optionally about 1.2% alginate.

In various embodiments, the culture medium is complete medium.

In various embodiments, the method further includes transporting the storage container to a hospital after storing for a period of time. Such time period may be one as disclosed herein.

In various embodiments, the method further comprises maintaining the temperature in the semipermeable membrane, or in the culture chamber, or in the storage container below 10° C., for example, from about 0° C. to about 4° C. and ranges in between, or at about 4° C.

In various embodiments, the CO₂ level, or the 02 level, or the humidity, or any combination thereof in the semipermeable membrane, or the culture chamber, or the storage container is/are not artificially controlled.

In various embodiments, the method further comprises dissolving the alginate gel prior to implantation, for example, by a method comprising exposing the encapsulated tissue(s) or organ(s) to a chelating solution. In further embodiments, the chelating solution comprises, consists essentially of, or further consists of a sodium citrate solution or an Ethylenediaminetetraacetic acid (EDTA) solution or a solution comprising both sodium citrate and EDTA. Additionally or alternatively, the alginate gel may be removed mechanically, such as by a manual removal.

In various embodiments, the method further comprises disassembling the semipermeable membrane, for example prior to implantation.

In various embodiments, the tissue(s) or organ(s) proceeded using a method as disclosed herein, for example those viable for implantation as provided, reduces or reduce release of inflammatory factor(s), for example, compared to a tissue or an organ stored without the encapsulation in an alginate gel, or in a semipermeable membrane, or both in an alginate gel and in a semipermeable membrane. In further embodiments, the inflammatory factor(s) comprises, consists essentially of, or further consists of nitric oxide, or one or more of cytokines, or both nitric oxide and one or more of cytokines.

In various embodiments, the tissue(s) or organ(s) processed using a method as disclosed herein, for example those viable for implantation as provided, maintains or maintain a cell viability of at least about 60%, or at least about 65%, or at least about 70%, or at least about 75%, or at least about 80%, or at least about 85%, or at least about 90%, or at least about 95% or higher of that prior to the encapsulating step or after the obtaining step (i.e., a step comprising, consisting essentially of, or consisting of obtaining the tissue(s) or organ(s) for example from a subject).

In various embodiments, the method further comprises injecting a buffer, such as a saline, into the alginate gel or the culture chamber or the alginate gel in the culture chamber prior to injecting the culture medium. In further embodiments, the buffer, such as a saline, reduces or removes soluble calcium or barium.

In a non-limiting example of the method as disclosed herein, a tissue is placed in a dialysis apparatus serving as a semipermeable membrane chamber (such as Tube-O-DIALYZER™, G-Bioscience, MO, USA) and filled with an alginate solution. In further embodiments, the alginate solution comprises or consists essentially of 1.2% alginate. Additionally or alternatively the dialysis apparatus is equipped with a 50 kDa cut-off semipermeable membrane. In yet further embodiments, the alginate solution is cross-linked/gelled by a calcium solution or a barium solution or both, optionally washed with normal saline, and then stored in complete medium.

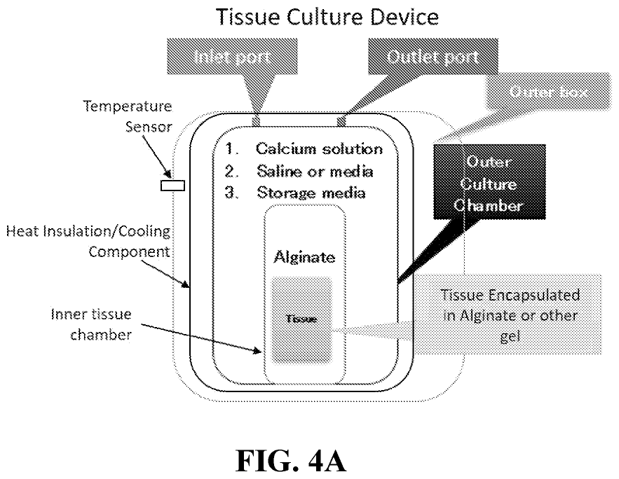

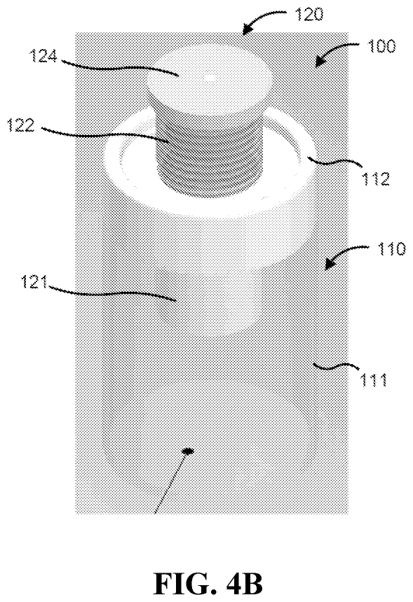

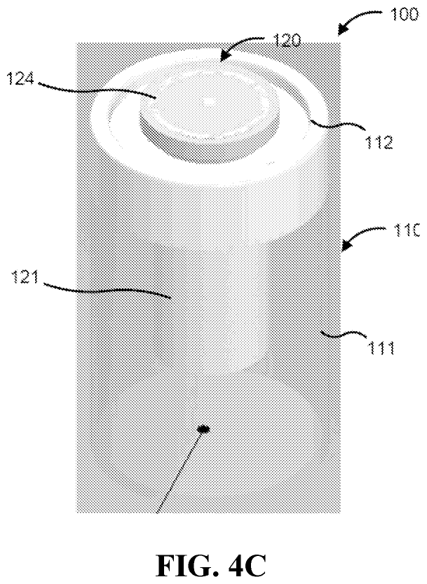

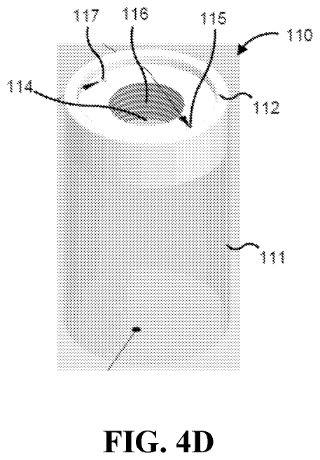

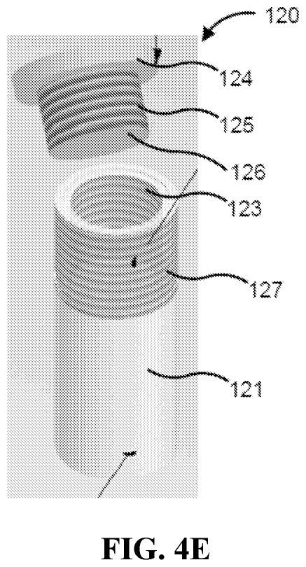

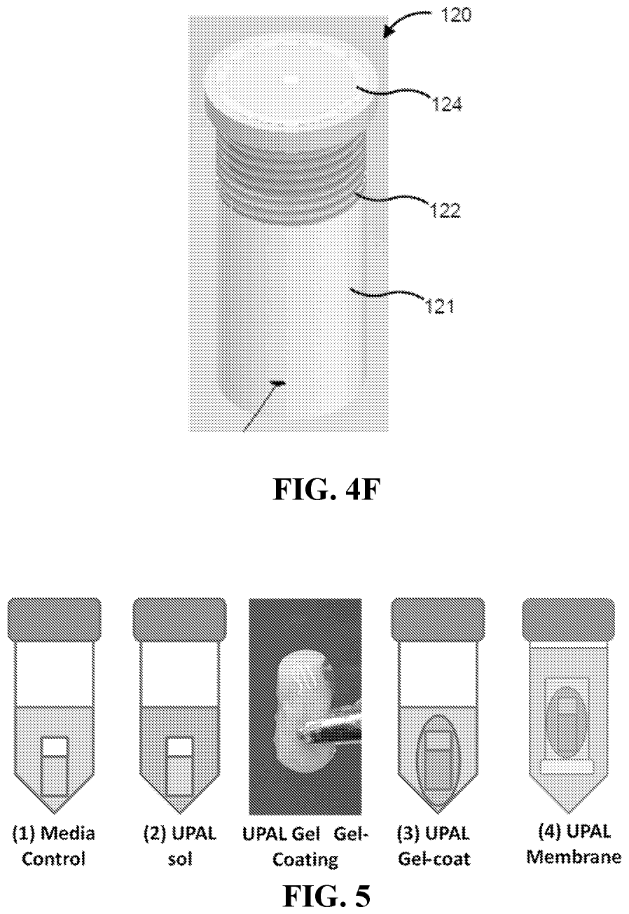

In another aspect, the disclosure provides a tissue transport system. For example, FIG. 4A shows a tissue culture device that can form part of a tissue transport system. The tissue culture device includes an outer chamber (also referred to herein as “culture chamber”) defining an inlet structured to allow inflow of a fluid, and an outlet structured to allow outflow of the fluid therethrough. In some embodiments, instead of having separate inlet and outlet ports, the outer chamber may have a single port which can be selectively used for communicating the fluid into or out of the outer chamber. In some embodiments, the fluid may include at least one of an alginate solution, a calcium solution, a barium solution, saline, culture media, or storage media. The outer chamber is enclosed, i.e., defines a closed internal volume.

The tissue culture device also includes an inner chamber (also referred to herein as “tissue chamber”) disposed within the outer chamber. The inner chamber has at least one wall formed from a semipermeable membrane (e.g., a portion of a wall, a cap, or a base formed from the semipermeable membrane). In various embodiments, the semipermeable membrane comprises, or consists essentially of, or alternatively consists of a mesh. In some embodiments, the semipermeable membrane may be a bag strainer structure or a cell strainer like structure. For example, the inner chamber may include a bag formed from a semipermeable membrane or mesh, such that the tissue is placed within the bag, and the bag is then disposed within the outer chamber. In some embodiments, the inner chamber may include walls formed from a non-porous material (e.g., plastics, polymers, or metals) and a cap of the inner chamber may at least partially be formed from the semipermeable membrane. The inner chamber is enclosed (i.e., defines a closed internal volume) and is configured to hold or contain a tissue therewithin, for example, for preserving or transporting tissue and contains the alginate gel which surrounds or encapsulates the tissue or organ. In some embodiments, after placing the tissue to be preserved/transported into the inner chamber, an alginate solution is added to the inner chamber. For example, the tissue may be immersed in an alginate solution and placed in the inner chamber, or the inner chamber containing the tissue may be filled with the alginate solution. Then a calcium solution or a barium solution or both is or are communicated into the outer chamber via the inlet. In other embodiments, the calcium solution or a barium solution both is or are communicated into the inner chamber before disposing the inner chamber into the outer chamber (e.g., by immersing the inner chamber into a container filled with the calcium solution and/or a barium solution), and once the alginate or other polymer network that encapsulates the tissue is formed within the inner chamber, the inner chamber is disposed into the outer chamber. In some embodiments, the tissue within the inner chamber may be immersed in a solution, such as one or more of an alginate solution, a calcium solution, a barium solution, or a culture medium. In further embodiments, the solution may be removed or replaced without releasing the tissue from the inner chamber.

While alginate gel is generally described herein, it should be appreciated that the tissue may be encapsulated in any other cross-linking gel, for example, a hydrogel or hydrophilic polymeric network, for example, agarose, methylcellulose, hyaluronan, elastin like polypeptides, polyethylene glycol (PEG), poly(N-hydroxyethyl methacrylate) (PHEMA), 2-hydroxyethyl methacrylate (HEMA), methacrylic acid (MAA), PEG-PEGMA, carboxymethyl cellulose (CMC), polyvinylpyrrolidone (PVP), acrylamide/acrylic acid copolymer, linear cationic polyallylammonium chloride, poly(N-isopropyl acrylamide) (PNIPAM), chitosan, acrylate-modified PEG and acrylate-modified hyaluronic acid, amine end-functionalized 4-arm star-PEG, any other suitable hydrogel or polymer or combination thereof. In some embodiments, a gel as used herein depolymerizes partially or completely in vivo, for example, by contacting with a solution, such as an alginate gel dissolving in a chelating solution. Additionally or alternatively, a gel as used herein permits flow of culture medium, a calcium solution, a barium solution, or a chelating solution therethrough. The gel that encapsulates the tissue (e.g., the alginate gel or any other gel or polymer described herein) provides the benefit of protecting the tissue disposed within the inner chamber, until the tissue is used. Moreover, the semipermeable membrane or mesh that forms at least a portion of the inner chamber prevents the alginate gel or any other gel or polymer that encapsulates the tissue from leaking out of the inner chamber while allowing the fluid (e.g., a calcium solution, a barium solution, saline, culture media, or storage media) from flowing through the semipermeable membrane or mesh so as to be in fluid communication with the internal volume of the inner chamber.

In various embodiments, a gel as disclosed herein (such as an alginate gel) may be dissolved by a method comprising, consisting essentially of, or alternatively consisting of exposing the gel to an chelating solution as disclosed herein (such as an EDTA solution or a sodium citrate solution. Additionally or alternative, a gel as disclosed herein may be dissolved by a method comprising, consisting essentially of, or alternatively consisting of exposing the gel to an excessive amount of saline solution.

In some embodiments, the semipermeable membrane may have a porosity (e.g., a pore size of at least about 250 pm, for example, about 0.22 μm to about 1,000 about 0.22 μm to about 100 about 100 μm to about 1,000 and any ranges in between) structured to prevent leakage of the alginate solution or the alginate gel from within the inner chamber while allowing the fluid to be communicated into therethrough into the inner chamber. In some embodiments, the inner chamber may include a dialysis cassette, for example, the dialysis cassette available under the tradename SLIDE-A-LYZER available from SIGMA ALDRICH®.

If not already present within the outer chamber, the inner chamber is thereafter placed in the outer chamber having the inlet port and the outlet port. The alginate solution may thereafter be gelled by adding a calcium solution or a barium solution or both to the inlet port of the culture chamber. After gelation, the calcium solution or the barium solution or both is or are washed by injecting saline or culture media, for example, by expelling the calcium solution or the barium solution or both from the outlet of the outlet chamber.

The tissue culture device or system also includes an enclosed storage container (also referred to herein as “box”) within which the outer chamber and thereby, the inner chamber is disposed within an internal volume defined by the storage container. For example, after washing the alginate gel from the outer chamber, the outer chamber is thereafter placed in the storage container. In some embodiments, a temperature sensor is operably coupled to the storage container and configured to measure a temperature within the storage container, for example, to ensure the tissue is maintained at a desired temperature during transportation (e.g., less than 10 degrees Celsius). In some embodiments, the tissue culture device may also include at least one of a heat insulation component or a cooling component disposed within the storage container. The heat insulation component or the cooling component is configured to maintain a temperature within the storage container and/or the outer chamber below 10° C.

The storage container may be stored for up to 4-8 weeks or shipped to a hospital for transplantation or both stored for up to 4-8 weeks and shipped to a hospital for transplantation. In the operating room, the storage media is replaced with a dissolving buffer for a few minutes. Thereafter, the inner chamber is disassembled, and the tissue encapsulated in alginate gel is transferred to an EDTA or a sodium citrate solution to dissolve the alginate gel. Once the alginate gel is dissolved, the tissue is recovered and washed with saline in preparation for transplant into a subject.

FIGS. 4B-4F are various views of a tissue culture device 100 and various components of the tissue culture device 100, according to another embodiment. The tissue culture device 100 includes an outer chamber 110 or culture chamber and an inner chamber 120 or tissue chamber that is at least partially disposed inside the outer chamber 110.

As shown in FIG. 4D, the outer chamber 110 includes a fluid containment portion 111 and an outer chamber cap 112 coupled to a top end of the fluid containment portion 111. The fluid containment portion 111 defines an internal volume structured to receive at least a portion of the inner chamber 120, and to contain a fluid (e.g., media, saline, calcium solution, washing solution, chelating solution, etc.). As shown in FIGS. 4B-4D, the fluid containment portion 111 may define a circular cross-section. In other embodiments, the fluid containment portion 111 may have a square, rectangular, oval, elliptical, polygonal, or any other suitable cross-sectional shape. In some embodiments, the fluid containment portion 111 may be formed from a transparent or translucent material (e.g., acrylic, plastics, glass, etc.)

The outer chamber cap 112 may be removably or fixedly coupled to the top end of the fluid containment portion 111. In some embodiments, the outer chamber cap 112 may be removably coupled to the containment portion 111, for example, via threads, a friction-fit, snap-fit, interference-fit, or any other suitable coupling mechanism. The outer chamber cap 112 defines an aperture 114 therethrough, the aperture 114 being configured to receive at least a portion of the inner chamber 120 therethrough. An inlet port 115 for communicating the fluid into the fluid containment portion 111, and an outlet port 117 for communicating the fluid out of the fluid containment portion 111 are defined through the outer chamber cap 112. In some embodiments, the inlet port 115 and the outlet port 117 may be defined between the aperture 114 and an outer periphery of the outer chamber cap 112, opposite to each other. Threads 116 may be defined on an inner wall of the aperture 114 and configured to mate with corresponding mating threads defined on the inner chamber 120, as described herein.

As shown in FIGS. 4E-4F, the inner chamber 120 includes a tissue containment portion 121 and an inner chamber cap 122 coupled to a top end of the tissue containment portion 121. The tissue containment portion 121 defines an internal volume structured to hold a tissue therewithin (e.g., any of the tissue described herein.) In some embodiments, at least a portion of the tissue containment portion 121 is formed from a semi-permeable membrane (e.g., any of the semipermeable membranes described herein or a mesh such as a medical grade mesh). For example, only a base of the tissue containment portion 121 may be formed completely or at least partially (such as at least about 10%, at least about 20%, at least about 30%, at least about 40%, at least about 50%, at least about 60%, at least about 70%, at least about 80%, at least about 90%, at least about 95%, or more) from the semipermeable membrane, only the walls of the tissue containment portion 121 may be formed completely or at least partially (such as at least about 10%, at least about 20%, at least about 30%, at least about 40%, at least about 50%, at least about 60%, at least about 70%, at least about 80%, at least about 90%, at least about 95%, or more) from the semipermeable membrane, or both of the walls and the base of the tissue containment portion 121 may be formed completely or at least partially (such as at least about 10%, at least about 20%, at least about 30%, at least about 40%, at least about 50%, at least about 60%, at least about 70%, at least about 80%, at least about 90%, at least about 95%, or more) from the semipermeable membrane. In some embodiments, the tissue disposed within the tissue containment portion 121 is encapsulated in alginate gel.

An inner chamber cap 124 is coupled to a top end of the tissue containment portion 121. For example, a first set of threads 123 may be defined on an inner surface of the top end of the tissue containment portion 121. The inner chamber cap 124 may include a projection 126 extending from a base of the inner chamber cap 124 towards the tissue containment portion 121. A set of mating threads 125 may be defined on a radially outer surface of the projection 121 and configured to mate with the first set of threads 123 so as to couple the inner chamber cap 124 to the tissue containment portion 121.

A second set of threads 127 may also be defined on a radially outer surface of the top end of the tissue containment portion 121 and are configured to mate with the threads 116 defined on the inner wall of the aperture 114. The tissue containment portion 121 is configured to be inserted through the aperture 114 of the outer chamber cap 112 into the fluid containment portion 111 until the second set of threads 127 mate with the threads 116 so as to secure the inner chamber 120 to the outer chamber 110. In some embodiments, a cross-sectional width (e.g., diameter) of the inner chamber cap 124 is larger than a cross-sectional width (e.g., diameter) of the aperture 114 such that the cap 124 cannot be inserted through the aperture 114 and remains disposed outside the outer chamber 110 when the tissue containment portion 121 is disposed inside the fluid containment portion 111. In some embodiments, the first set of threads 123 that couple to the mating threads 125 of the inner chamber cap 124, may be oriented in the opposite direction to the second set of threads 127 that couple to threads 116 of the aperture 114, so as to prevent uncoupling of the inner chamber cap 124 from the tissue containment portion 121, when the tissue containment portion 121 is being unthreaded from the outer chamber cap 112. In operation, a user may hold the inner chamber 120 by the inner chamber cap 124 for coupling the inner chamber 120 to, or uncoupling the inner chamber 120 from the outer chamber 110. The tissue culture device 100 be disposed within a storage container or box, as described herein, for transportation.

In one aspect, provided is a system comprising, consisting essentially of, or further consisting of an outer chamber (which is also referred to herein as a culture chamber) comprising an inner chamber equipped with a semipermeable membrane (which is also referred to herein as a tissue chamber or a semipermeable membrane chamber). In various embodiments relating to any aspect as disclosed herein, a wall of the inner chamber may be formed completely or at least partially (such as at least about 10%, at least about 20%, at least about 30%, at least about 40%, at least about 50%, at least about 60%, at least about 70%, at least about 80%, at least about 90%, at least about 95%, or more) from the semipermeable membrane.

In some embodiments, the semipermeable membrane has a pore size of at least about 250 pm, for example, about 0.22 μm to about 100 μm and ranges in between, about 0.22 μm to about 1000 μm and ranges in between, or about 100 μm to about 1000 μm and ranges in between.

In some embodiments, the semipermeable membrane has a mesh opening rate of about 1% to about 50% and ranges or percentages in between, for example about 2%.

In some embodiments, MWCO of the semipermeable membrane is about 50 kDa or about 200,000 kDa.

In various embodiments, the inner container (which is also referred to herein as the inner chamber) can be closed after filling with an alginate solution and fixed in the outer container (which is also referred to herein as the outer chamber). Additionally or alternatively, the inner container/chamber can be removed from the outer container/chamber to recover the tissue in an operation room for transplantation.

In various embodiments, the outer container (which is also referred to herein as the outer chamber) comprises inlet and outlet ports to infusing crosslink solution (i.e., a solution that gels an alginate solution, such as a calcium solution a barium solution or both, e.g. a calcium chloride solution or a barium chloride solution) or dissolving solution (i.e., a solution that dissolves an alginate gel, such as a chelating solution, for example, an EDTA solution or a sodium citrate solution or a solution comprising both EDTA and sodium citrate). Accordingly, without opening the inner container (which is also referred to herein as the inner chamber), gelling of alginate or dissolving of alginate can be achieved aseptically.

In one aspect, provided herein is a newly designed embedded system or apparatus with alginate gel and an optional semipermeable membrane.

In some embodiments, the system or the apparatus provides tissues some protection from mechanical damage during tissue transport to the clinical site. In some embodiments, the tissue is embedded in the apparatus or the system with alginate,

In some embodiments, the system or the apparatus provides tissues some protection from mechanical damage during tissue transport to the clinical site. In some embodiments, the tissue is embedded in the apparatus or the system with alginate, stored at 4° C., and transferred from a first location, such as organ bank, to a second location, such as a hospital.

In some embodiments, embedding, storage, and transfer is accomplished using a single apparatus (i.e., the system described herein). In some embodiments, after placing tissues in the apparatus or the system, alginate solution and calcium solution are added to the apparatus or the system to surround or encapsulate the tissue. In some embodiments, the tissue is placed in a semipermeable membrane chamber, the alginate solution, and the calcium solution (or alternatively the barium solution or the solution comprising or consisting essentially of a bariums salt and a calcium salt) are added to the chamber. In some embodiments, alginate is gelled by adding a calcium solution or a barium solution, or both to the chamber. In some embodiments, after gelation, the alginate gel is washed by injecting saline or culture media to remove excess calcium or barium (i.e., soluble calcium or barium).

In some embodiments, the semipermeable membrane chamber (i.e., the inner chamber) is locked or fixed in a culture chamber (i.e., an outer chamber). In some embodiments, two double-layer chambers is placed in an outer box (i.e., a storage container or box) with a temperature sensor.

In some embodiment, the tissue is then stored for at least about 2 weeks, such as at least about 4 weeks, at least about 5 weeks, at least about 6 weeks, at least about 7 weeks, at least about 8 weeks, at least about 9 weeks, at least about 10 weeks, or longer. Additionally or alternatively, the tissue is stored for up to 4 weeks, or up to 6 weeks, or up to 8 weeks, or up to 10 weeks. In some embodiment, the tissue is then stored for about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 5 weeks to about 6 weeks, about 5 weeks to about 7 weeks, about 5 weeks to about 8 weeks, about 5 weeks to about 9 weeks, about 5 weeks to about 10 weeks, about 6 weeks to about 7 weeks, about 6 weeks to about 8 weeks, about 6 weeks to about 9 weeks, about 6 weeks to about 10 weeks, or any ranges in between of each. In some embodiments, the tissue is preserved for up to 4-8 weeks.

In some embodiments, the outer box with two chamber is shipped to hospital. In some embodiments, after arrival at the hospitals, the alginate gels are dissolved with sodium citrate or EDTA in the apparatus or the system in an operating room. In some embodiments, in the operating room, storage media (for example, the culture medium) is replaced with dissolving buffer for a few minutes. In some embodiments, the semipermeable membrane chamber is de-assembled and the tissue with alginate gel is transferred to the EDTA solution to dissolve the alginate gel. In some embodiments, the tissue is recovered from the alginate gel and washed with saline for transplant.

The tissue transport system provides several advantages over a standard liquid-based tissue storage system. The tissue preservation condition is improved by embedding in alginate gel. Tissues are protected from shock, vibration or shaking during transporting tissues to hospitals. As such, tissue damage by transport can be prevented. In addition, as shown herein, cell viability is improved and inflammatory factors, such as cytokines and nitric oxide are suppressed. One enclosed system covers the storage and transport stage.

In some embodiments, the tissue transport system and/or tissue culture device may be provided in a kit. For example, the kit may include (for example, comprise, consist essentially of, or alternatively consist of) the tissue culture device including the outer chamber (e.g., the outer chamber 110 or the inner chamber described in FIG. 4A), and the inner chamber (e.g., the inner chamber 120 or the inner chamber described in FIG. 4A). The kit may also include (for example, further comprise) the storage container or box, for example, as described with respect to FIG. 4A, that may have a heating insulating and/or cooling component, and a temperature sensor. The kit may also include (for example, further comprise) a container or vial containing alginate solution or any other hydrogel solution, a container or vial containing a calcium solution or a barium solution or both, a container or vial containing a calcium salt or a barium salt or both, a container or vial filled with a washing solution (e.g., a saline solution or any other solution as described herein), and/or a culture medium (e.g., any of the culture mediums described herein). The kit may also include written instruction or downloadable instructions for using the tissue culture device as described herein. In various embodiments, a kit as disclosed herein comprises or consists essentially of a tissue culture device as disclosed herein and an optional instruction for use. In various embodiments, a kit as disclosed herein further comprises one or more of the following: alginate or any other hydrogel monomer optionally in a solution, a calcium solution, a barium solution, a calcium salt, a barium salt, a solvent, a washing solution, or a culture medium.

### EXAMPLES

The following examples are intended to illustrate but not limit the disclosure.

### Example 1

### In Vitro Evaluation of the Effects of Ultra-Purified Alginate on the Performance of Osteochondral Allografts Harvested from Pig Knees

### Methods

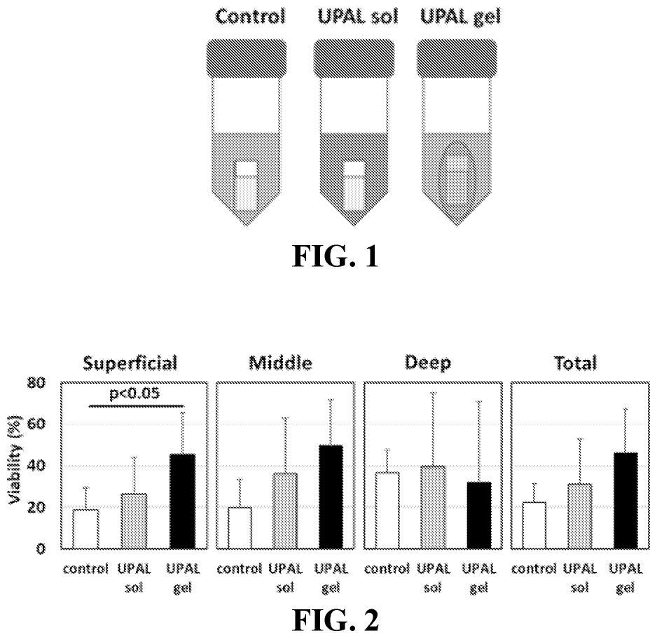

Graft preparation and storage conditions—Osteochondral cores (n=25) were harvested from 10 knees of 5 mature female pigs obtained from a slaughter house within 48 hours after sacrifice. Under sterile conditions, osteochondral cores were harvested by coring with a custom 4.0-mm bit drill from either the medial or lateral femoral condyle. Before storage, the core bone was trimmed to a 4:1 bone-to-cartilage-thickness ratio. The subchondral bone was subjected to pulse lavage with air to remove marrow elements and subsequently thoroughly rinsed with normal saline. Each osteochondral core was stored overnight in DMEM/F12 with 10% fetal bovine serum (FBS) and then placed into an individual sterile 5-mL culture tube for storage at 4° C. according to the condition assigned to each core as follows (FIG. 1): (1) the control group—stored in 1.5 mL complete medium (DMEM/F12+10% FBS); (2) the UPAL solution (sol) group—stored in 1.5 ml UPAL solution (1.2%) dissolved in complete medium; (3) the UPAL gel group—encapsulated with 1.2% UPAL gel dissolved by normal saline and stored in 1.5 ml complete medium. After 28 days of storage, each sample was warmed for 24 hours at 37° C.; the full-thickness cartilage was then removed from the bone to perform the evaluations described below.

Chondrocyte viability—Chondrocyte viability (n=4) throughout the full-thickness cartilage depth was analyzed using a LIVE/DEAD kit and imaged using confocal laser microscopy. For vertical profiles, viability was calculated for the overall cartilage thickness as well as zones of cartilage: superficial (top 15%), middle (35%), and deep (50%).

Proteoglycan (PG) content—The PG content (n=6) in each cartilage core was measured using the dimethylmethylene blue (DMMB) assay (normalized by wet weight).

PG synthesis—The incorporation of radiolabeled ³⁵S-sulfate into sulfated PGs (n=6) was measured as an indicator of PG synthesis by a rapid Alcian blue filtration assay (Masuda, et al., Anal Biochem. 1994:167).

PG turnover—The number/amount of ³⁵S-PGs in the media and cartilage digests (n=6) was measured and ³⁵S-PGs remaining in the tissue over the total amount of ³⁵S-PGs synthesized was assessed as PG turnover (O'Driscoll, et al., J Bone Joint Surg Am. 1986:1017).

Nitric oxide (NO) and matrix metalloproteinase-3 (MMP-3) concentration—The storage media collected at day 28 was assayed for total NO (n=6) and MMP-3 (n=4) concentration using commercially available assay kits.

Histology—Mid-vertical sections (5 μm) of each OCA (n=7) were stained with either hematoxylin and eosin or safranin-O. An observer blinded to this experiment analyzed the histologic sections and graded them using a modified O'Driscoll histologic scoring system (Aota, et al., Spine 2005:722).

Statistical analyses—All data are expressed as mean±standard deviation (SD). The data were statistically analyzed using two way-repeated analysis of variance (ANOVA) or one-way ANOVA for comparisons. P values less than 0.05 are regarded as statistically different.

### Results

Chondrocyte viability—Viability of the chondrocytes in the surface area of the UPAL gel group (45.4±20.1%) was significantly higher than that of the control group (18.6±10.7%, p<0.05; FIG. 2).

Proteoglycan (PG) content—No significant differences were found in PG content at day 28 (Control: 276.0±40.5 μg/mg, UPAL solution: 329.1±54.0 μg/mg, UPAL gel: 314.2±49.3 μg/mg).

PG synthesis— PG synthesis in the UPAL gel group (33192.8±15906.4 CPM/mg) was significantly higher than that in the control group (12748±2699 CPM/mg, p<0.05) at day 28. There was a significant decrease in ³⁵S incorporation at day 28 compared to day 1 (2073811±496258 CPM/mg, p<0.01) in all groups.

PG turnover—No significant differences were found in the ³⁵S-PGs remaining in the tissue at day 28.

Nitric oxide (NO) and matrix metalloproteinase-3 (MMP-3) concentration in the storage media—Total NO production in the control group (3.8±2.4 μM/mg) was significantly higher than that of the UPAL solution (1.0±0.8 μM/mg, p<0.01) and UPAL gel groups (1.5±0.8 μM/mg, p<0.01) at day 28. No significant differences were found in MMP-3 levels among the three conditions in the storage medium at day 28.

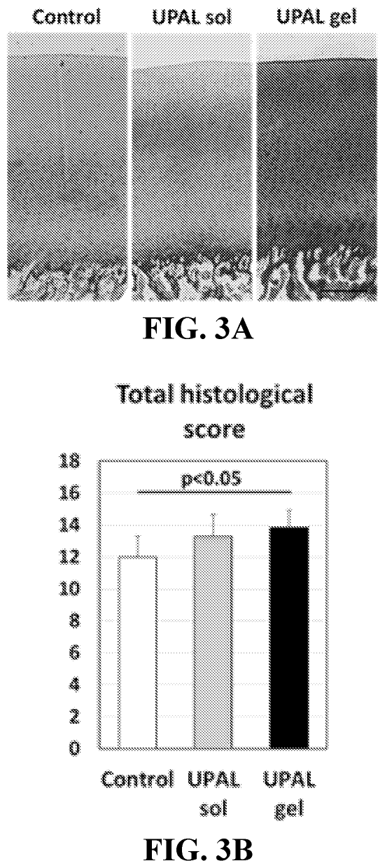

Histology—While the cartilage in the control group showed poor safranin-O staining matrix and low cellularity, the OCA in the UPAL gel group had abundant matrix staining and near normal cellularity. The total histological score in the UPAL gel group (13.9±1.1) was significantly higher than that of the control group (12.0±1.3, p<0.01; FIG. 3).

### Example 2

Example 1 showed that the use of ultra-purified alginate (UPAL) gel encapsulation was effective for improving the quality of osteochondral grafts after 28 days of storage. It is important to extend the storage period to increase the availability of grafts for clinical use. First, the instant Example 2 showed successful extension of the storage period for cartilage for up to six weeks. In addition, the application was extended to other tissues (meniscus, tendon, and cornea). Second, to establish improved storage conditions with UPAL, the effects of differing gelling conditions of UPAL were studied on cell viability during the extended storage period. It is worth noting that the endpoint of this experiment is one and one-half times (6W) that of the current protocol for tissue preservation. Importantly, the extension of the viable tissue storage period provides more availability of graft tissues. The use of UPAL gel during the tissue storage period is beneficial for cell viability and tissue structure maintenance during the storage period and also provides physical protection as a packing material during shipment.

### Methods

Graft preparation and storage conditions—Osteochondral cores, meniscus, patellar tendon, and cornea were harvested from young mini-pigs (6-months old). Under sterile conditions, osteochondral cores were harvested by coring with a custom 4.0-mm drill bit from either the medial or lateral femoral condyle. The subchondral bone was subjected to pulse lavage with air to remove marrow elements and subsequently thoroughly rinsed with normal saline. Before storage, the core bone was trimmed to a 4:1 bone-to-cartilage-thickness ratio. Tissues from medial and lateral menisci were punched out using skin pinch (4 mm) and patellar tendons were dissected aseptically and minced to 5×10 mm. Corneal tissues with the vitreous body were also dissected, and 5×10 mm implants were prepared. Each graft tissue was stored overnight in DMEM/F12 with 10% fetal bovine serum (FBS) and then placed into an individual sterile 5-mL culture tube for storage at 4° C. according to the condition assigned to each core as follows (FIG. 5): (1) the control group—stored in 3 mL complete medium (DMEM/F12+10% FBS); (2) the UPAL solution (sol) group—stored in 3 ml UPAL solution (1.2%) dissolved in complete medium; (3) the UPAL gel-coated group—encapsulated with 1.2% UPAL gel (approximately 1 ml UPAL, cross-linked by calcium solution and washed with normal saline), and stored in 3 ml complete medium; and (4) the UPAL membrane group: Tissues, prepared as described in the UPAL gel-coated group, were placed in the dialysis apparatus (Tube-O-DIALYZER™, G-Bioscience, MO, USA) equipped with 50 kDa cut-off semipermeable membrane) and filled with 1.2% alginate solution. Tubes containing one graft were immersed in 0.1 M CaCl₂ solution for 30 min to facilitate gelling. Tubes were then washed in saline and placed in 50 ml culture tubes filled with 40 ml complete media. Articular cartilage, meniscus, and Achilles tendon were stored for six weeks, whereas corneal tissues were stored for two weeks, all at four degrees.

After six weeks of storage, each sample was warmed for 24 hours at 37° C.; the tissues were then placed in 30 mM EDTA solution for 5 minutes to dissolve the alginate gel. Cell viability was analyzed using a LIVE/DEAD kit and imaged using confocal laser microscopy. For vertical profiles, viability was calculated using the Otsu-thresholding method, followed by the particle analysis function of the Image-J program. Viability was expressed as the viability at the endpoint/the original cell viability.

Statistical analyses—All data are expressed as mean±standard error (SE). The data were statistically analyzed using the one-way analysis of variance (ANOVA) or Kruskal-Wallis test adjusted by the Bonferroni correction for multiple tests. P values less than 0.05 are regarded as statistically different.

### Results

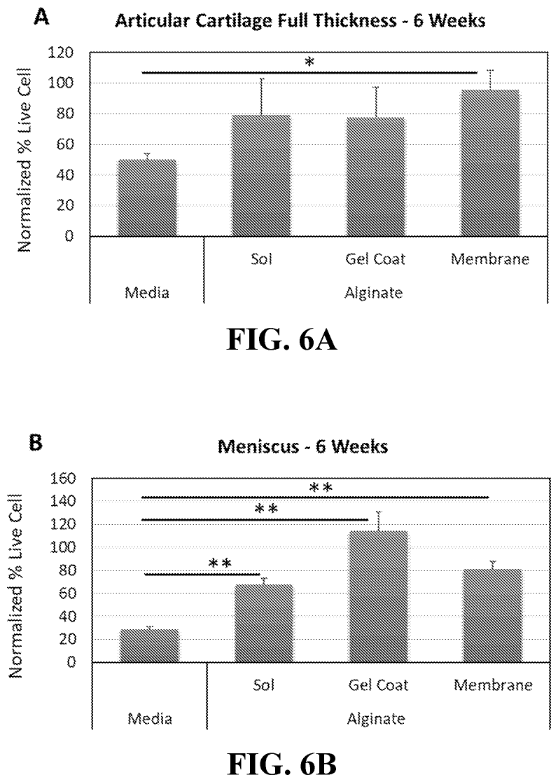

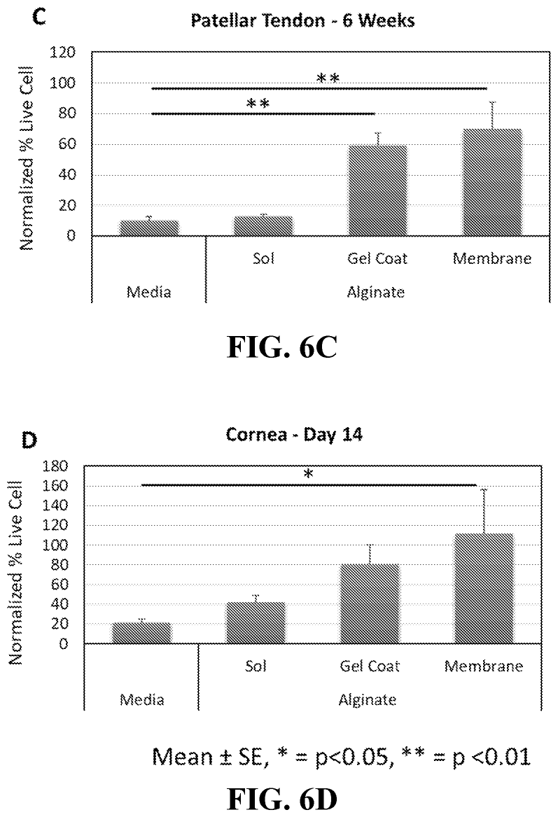

Articular Cartilage—At six weeks, the viability of chondrocytes in the surface area of the UPAL membrane group was significantly higher than that of the media group (p<0.05; FIG. 6A). At six weeks, the viability of chondrocytes in the UPAL sol group and UPAL gel-coated group showed a tendency to be higher, but statistical significance was not achieved because of sample size.

Meniscus—Viability of cells in the menisci had significantly dropped in the media group after six weeks (FIG. 6B). The UPAL sol, UPAL gel-coated and UPAL membrane groups showed significantly higher cell viability than that of the media group.

Patellar tendon—As seen in the meniscus, the viability of cells in the patellar tendon had significantly dropped in the media group after six weeks. Cell viability in the UPAL gel-coated and UPAL membrane groups was significantly higher than that in the media group (FIG. 6C).

Cornea—After two weeks, corneal tissue cell viability in the UPAL membrane group was significantly higher than that of the media group (FIG. 6D). Cell viability of the UPAL membrane group was not significantly different from that of the original tissue.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

Although the disclosure has been described with reference to the above examples, it will be understood that modifications and variations are encompassed within the spirit and scope of the disclosure. Accordingly, the disclosure is limited only by the following claims.

1-40. (canceled)

41. A method of preservation of a tissue, the method comprising:
encapsulating a tissue in an alginate gel; and
exposing the gel-encapsulated tissue to a culture medium, the tissue comprising one or more of an osteochondral tissue, an articular cartilage free of any subchondral bone, a fibrocartilage tissue, a tendon, and a cornea.

42. The method of claim 41, wherein the gel-encapsulated tissue is in the culture medium for a time period selected from about 24 hours to about 8 weeks, for about 28 days, for at least about 6 weeks and ranges in between.

43. The method of claim 41, further comprising:
encapsulating the tissue in a semipermeable membrane; and
disassembling the semipermeable membrane after the exposure with the culture medium.

44. The method of claim 43, wherein the tissue is encapsulated in the gel after being encapsulated in the semipermeable membrane.

45. The method of claim 43, wherein the tissue is encapsulated in the gel prior to being encapsulated in the semipermeable membrane.

46. The method of claim 41, wherein the alginate gel is ultra-purified alginate (UPAL) gel present at a concentration of about 0.5% to about 2.0% or about 1.2% and ranges in between.

47. The method of claim 41, further comprising:
dissolving the alginate gel after the exposure with the culture medium, wherein the culture medium is a complete medium.

48. The method of claim 41, further comprising:
exposing the encapsulated tissue to a chelating solution optionally selected from a sodium citrate solution or an Ethylenediaminetetraacetic acid (EDTA) solution or a solution comprising both sodium citrate and EDTA.

49. The method of claim 41, whereby
the encapsulated tissue reduces release of inflammatory factors optionally selected from a cytokine or nitric oxide, optionally compared to a tissue stored without the encapsulation, and
the encapsulated tissue prior to the implantation maintains a cell viability of at least about 60%, or at least about 65%, or at least about 70%, or at least about 75%, or at least about 80%, or at least about 85% or higher of that prior to the encapsulating step.

50. A tissue transporting system comprising:
a tissue chamber partially or fully formed from a semipermeable membrane and configured to contain tissue to be transported and an alginate solution, the tissue chamber being enclosed;
a culture chamber having an inlet port and an outlet port, the culture chamber being configured to contain the tissue chamber, wherein upon addition of a calcium solution or a barium solution or both through the inlet port, the alginate solution gels and encapsulates the tissue, the culture chamber being enclosed; and
one or more of a transport box and a storage container sealed against continuous flow of gases or liquids and configured to contain the culture chamber.

51. The system of claim 50, wherein the alginate solution is an ultra-purified alginate solution.

52. The system of claim 50, wherein the alginate solution comprises about 0.5% to about 2.0% alginate, optionally about 1.2% alginate and ranges in between.

53. The system of claim 50, further comprising:
a temperature sensor disposed in the transport box or the storage container and configured to measure temperature of the culture chamber; and
the tissue chamber including an enclosed tissue chamber configured to encapsulate the tissue by the semipermeable membrane.

54. The system of claim 50, wherein the semipermeable membrane has one or more of: a pore size of at least about 250 pm, a pore size of about 100 μm to about 1000 μm, a pore size of about 0.22 μm to about 1000 μm, or about 0.22 μm to about 100 μm; a molecular weight cut-off (MWCO) at about 50 kDa or at about 200,000 kDa; or a mesh opening rate of about 1% to about 50%.

55. The system of claim 50, further comprising:
a heat insulation component configured to maintain the temperature in one or more of the transport box, the storage container, and the culture chamber below 10° C., optionally from about 0° C. to about 4° C., and further optionally at about 4° C.

56. The system of claim 50, further comprising:
a cooling component configured to maintain the temperature in one or more of the transport box, the storage container, and the culture chamber below 10° C., optionally from about 0° C. to about 4° C., and further optionally at about 4° C.

57. A tissue culture device, comprising:
an outer chamber defining an inlet structured to allow inflow of a fluid, and an outlet structured to allow outflow of a fluid therethrough, the outer chamber being enclosed; and
an inner chamber disposed at least partially within the outer chamber, the inner chamber having at least one wall formed from a semipermeable membrane, the inner chamber being enclosed and structured to hold a tissue therewithin.

58. The tissue culture device of claim 57, further comprising:
an enclosed storage container, the inner chamber disposed within an internal volume defined by the storage container; and
a temperature sensor operably coupled to the storage container and configured to measure a temperature within the storage container.

59. The tissue culture device of claim 57, wherein the fluid comprises at least one of an alginate solution, a calcium solution, a barium solution, saline, culture media, or storage media.

60. The tissue culture device of claim 57, wherein the semipermeable membrane has a molecular weight cut-off (MWCO) at about 50 kDa or about 200,000 kDa,
wherein the semipermeable membrane has a pore size of at least about 250 pm and in a range of about 0.22 μm to about 100 μm, or about 100 μm to about 1,000 μm, or about 0.22 μm to about 1,000 μm, and
wherein the semipermeable membrane has a mesh opening rate of about 1% to about 50%.
